(19) United States
(12) Patent Application Publication
(10) Pub. No.: US 2021/0353721 A1
(43) Pub. Date: Nov. 18, 2021

(54) METHODS AND COMPOSITIONS RELATING TO INHIBITING CARDIOVASCULAR CALCIFICATION VIA ANNEXIN A1

(71) Applicant: THE BRIGHAM AND WOMEN'S HOSPITAL, INC., Boston, MA (US)

(72) Inventors: Elena AIKAWA, Chestnut Hill, MA (US); Maximillian ROGERS, Boston, MA (US); Masanori AIKAWA, Chestnut Hill, MA (US)

(73) Assignee: THE BRIGHAM AND WOMEN'S HOSPITAL, INC., Boston, MA (US)

(21) Appl. No.: 17/265,238

(22) PCT Filed: Aug. 7, 2019
(86) PCT No.: PCT/US2019/045394

§ 371 Date: Feb. 2, 2021

Related U.S. Application Data
(60) Provisional application No. 62/715,428, filed on Aug. 7, 2018.

(51) Int. Cl. A61K 38/48; A61K 31/513; A61K 31/198; C07K 16/18; C12N 15/113

(52) U.S. Cl. CPC A61K 38/482; A61K 31/513; A61K 31/198; C12N 2310/14; C12N 15/113; C12Y 304/21037; C07K 2317/76; C07K 16/18

(57) ABSTRACT

The technology described herein is directed methods of treating an extracellular vesicle (EV)-associated disease or vascular calcification in subject by administration of an agent that reduces the levels or activity of Annexin A1 (ANXA1).

### CROSS-REFERENCE TO RELATED APPLICATIONS

This application claims benefit under 35 U.S.C. § 119(e) of U.S. Provisional Application No. 62/715,428 filed Aug. 7, 2018, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 22, 2019, is named 043214-093330WOPT_SL.txt and is 119,910 bytes in size.

### TECHNICAL FIELD

The technology described herein relates to methods and compositions for the treatment of vascular calcification, e.g., in extracellular vesicle (EV)-associated diseases.

### BACKGROUND

Cardiovascular cells secrete extracellular vesicles (EVs) that aggregate in the vascular system and form microcalcifications. These microcalifications contribute to the pathology of a number of diseases and promote both atherosclerotic plaque rupture and heart valve failure. The mechanisms that induce the binding and aggregation of EVs to each other have not been previously identified and currently, no anti-calcification drug therapies are available.

### SUMMARY

As described herein, the inventors have now discovered that the aggregation of the EV's is controlled by annexin A1 (ANXA1). Inhibition of ANXA1 reduces aggregation of EVs as well as calcification. Accordingly, methods and compositions relating to inhibition of ANXA1 are provided herein for the treatment of calcification as well as a number of EV-associated diseases, including autoimmune and neurodegenerative diseases and cancer.

In one aspect of any of the embodiments, described herein is a method of treating an extracellular vesicle (EV)-associated disease in subject, the method comprising: administering an agent that reduces the levels or activity of Annexin A1 (ANXA1) in a subject in need thereof In some embodiments of any of the aspects, the EV-associated disease is selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.

In one aspect of any of the embodiments, described herein is a method of reducing vascular calcification in subject, the method comprising: administering an agent that reduces the levels or activity of Annexin A1 (ANXA1) in a subject in need thereof. In some embodiments of any of the aspects, the subject has a disease selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.

In one aspect of any of the embodiments, described herein is a method for inhibiting or reducing trafficking of an extracellular vesicle (EV) from a cell, the method comprising: contacting the cell with an agent that reduces the levels or activity of ANXA1. In some embodiments of any of the aspects, the cell is a smooth muscle cell (SMC), a valvular interstitial cell (VIC), oligodendroglioma cell, endothelial cell, macrophage, monocyte, or cancer cell.

In some embodiments of any of the aspects, the agent is an inhibitor of ANXAl. In some embodiments of any of the aspects, the agent is a small molecule, nucleic acid, polypeptide, antibody reagent, or genome editing system. In some embodiments of any of the aspects, the nucleic acid is an inhibitory nucleic acid, silencing RNA (siRNA), microRNA (miRNA), or short hairpin RNA (shRNA). In some embodiments of any of the aspects, the siRNA sequence comrpises SEQ ID NO: 1 or 2. In some embodiments of any of the aspects, the antibody reagent is an anti-ANXA1 antibody reagent. In some embodiments of any of the aspects, the antibody reagent is an ANXA1 neutralizing antibody reagent. In some embodiments of any of the aspects, the ANXA1 neutralizing antibody reagent is an N-terminal ANXA1 neutralizing antibody reagent. In some embodiments of any of the aspects, the small molecule is a calcium chelator. In some embodiments of any of the aspects, the calcium chelator is ethylenediaminetetraacetic acid (EDTA).

In some embodiments of any of the aspects, the agent reduces ANXA1 loading into the extracellular vesicles. In some embodiments of any of the aspects, the agent is an inhibitor of dynamin-related protein 1 (DRP 1). In some embodiments of any of the aspects, the agent is mdivi-1 or is an inhibitory nucleic acid.

In some embodiments of any of the aspects, the agent induces cleaves of the N-terminal domain of ANXA1. In some embodiments of any of the aspects, the agent is an agonist of proteinase 3 or HLE.

In some embodiments of any of the aspects, the subject is a mammal. In some embodiments of any of the aspects, the subject is a human.

In some embodiments of any of the aspects, the method further comprises receiving the results of an assay that indicates that the subject has an increase in the level of microcalcifications before administering the agent that reduces the levels or activity of ANXA 1. In some embodiments of any of the aspects, the method further comprises receiving the results of an assay that indicates that the subject has an increase in the level or activity of ANXA1. In some embodiments of any of the aspects, the assay is selected from the group consisting of: an extracellular vesicle calcification assay; a vesicle binding assay; a vesicle tethering assay; a tissue non-specific alkaline phosphatase (TNAP) assay; an immunohistochemistry assay; mass spectrometry; proteomics; scanning electron microscopy; and transmission electron microscopy. In some embodiments of any of the aspects, the biological sample is blood, a vascular tissue, or a heart valve tissue.

### BRIEF DESCRIPTION OF THE DRAWINGS

FIGS. 1A-1D demonstrate that human cardiovascular tissue SMCs and VICs released EVs that aggregated in collagen extracellular matrix. (FIG. 1A) SMCs in calcified human carotid artery tissue and (FIG. 1B) VICs in calcified human aortic valve tissue transmission electron microscopy images (n=3 donors, with representative images shown; scale bars, 500 nm). Arrows in the top panels indicate EVs that likely budded from plasma membrane and arrows in the bottom panels indicate multivesicular bodies likely being released. (FIG. 1C) Aggregated EVs (arrows) in collagen extracellular matrix away from any cells in calcified human carotid artery and (FIG. 1D) in calcified human aortic valve tissues (n=3 donors, with representative images shown; scale bars, 100 nm).

FIGS. 2A-2C demonstrate that aggregated and calcifying EVs are likely precursors of microcalcifications in calcified human cardiovascular tissue. (FIG. 2A) Transmission electron microscopy images of aggregated and calcifying EVs (black arrows indicate EVs with membrane hydroxyapatite formation) in collagen extracellular matrix in human carotid artery and human aortic valve tissues (n=3 donors, with representative images shown; scale bars, 200 nm). (FIG. 2B) Density-dependent scanning electron microscopy images of aggregated microcalcification in human carotid artery and (FIG. 2C) human aortic valve tissue matrix; scale bars, 1 μm (n=5 donors with representative images shown).

FIGS. 3A-3C demonstrate that human cardiovascular EV protein composition was altered under osteogenic conditions. (FIG. 3A) Nanoparticle tracking analysis of size and abundance of human SMC- and VIC-derived EVs in conditioned media from cells cultured in NM or OM and used in proteomics analysis; data are mean±s.d. from 3 donors. (FIG. 3B) Transmission electron microscopy images of EVs isolated from SMC and VIC conditioned media and used in proteomics analysis (n=3 pooled donors, with representative images shown). (FIG. 3C) Proteomics volcano plot analysis for EVs derived from human SMC (n=9 donors) and VIC (n=7 donors) conditioned media. Plots show increased, insignificant, and decreased EV protein abundances, along with pie charts of total detected protein distribution in OM relative to control NM.

FIGS. 4A-4B demonstrate that human SMC- and VIC-derived EVs contained tethering proteins. (FIG. 4A) Enriched pathways based on significantly changed proteins in OM from EVs obtained from SMCs (n=9 donors) and VICs (n=7 donors). Shared pathways enriched in both SMC- and VIC-derived EVs indicated by blue nodes on the pathway networks and listed in the adjacent heat map. Connections in networks denote common proteins between pathways (more proteins equate to a thicker connection). Node sizes correspond to significance of enrichment (proportional to -log(q-value)). (FIG. 4B) Venn diagram of detected EV proteins increased in OM, with the left-most circle including proteins detected in SMC-derived EVs (n=9 donors) and the right-most circle including proteins detected in VIC-derived EVs (n=7 donors). Annexin proteins (ANXA1, ANXA2, ANXA5, ANXA6, ANXA7) detected in both SMC- and VIC-derived EVs and increased in OM in either or both indicated in dark text. Additional proteins detected and increased in OM in SMC-derived EVs (left-most circle), VIC-derived EVs (right-most circle), or both SMC- and VIC-derived EVs (overlapping region) indicated in lighter text.

FIGS. 5A-5C demonstrate that calcified human cardiovascular tissues and tethered EVs contained ANXA1. (FIG. 5A) ANXA1 immunohistochemistry in calcified (purple color) human carotid artery and aortic valve tissues. Scale bars, 20 μm; n=5 donors, with representative images shown. (FIG. 5B) Human carotid artery and aortic valve tissue ANXA1 immunofluorescence near aggregated microcalcifications (OsteoSense) in collagen matrix (CNA35-0G488). Scale bars, 20 μm; n=5 donors, with representative images shown. (FIG. 5C) Calcified human carotid artery and aortic valve tissue transmission electron microscopy of ANXA1 immunogold labeling (black dots) on tethered EVs (arrow) in collagen extracellular matrix. Scale bars, 100 nm; n=3 donors, with representative images shown.

FIGS. 6A-6C demonstrate that ANXA1 knockdown attenuated human SMC and VIC calcification. (FIG. 6A) Human SMCs and VICs ANXA1 mRNA levels and ANXA1 protein from cells cultured in control NM or OM and treated with control siRNA (control) or ANXA1 siRNA (ANXA1si). Data are mean±s.d. from 3 donors; ***P<0.001, **P<0.01,*P<0.05, analyzed by ANOVA. (FIG. 6B) TNAP activity in human SMCs and VICs in NM or OM with control siRNA or ANXA1si; n=3 donors, data are mean±s.d., ****P<0.0001, ***P<0.001, **P<0.01, *P<0.05, analyzed by ANOVA. (FIG. 6C) Confocal microscopy images of microcalcifications (Osteosense staining; DAPI) in SMCs and VICs cultured in NM or OM with control siRNA or ANXA1si; n=3 donors, data are mean±s.d., **P<0.01, *P<0.05, analyzed by ANOVA.

FIGS. 7A-7D demonstrate that calcium signaling dysfunction increased ANXA1 loading on SMC- and VIC-derived EVs. (FIG. 7A) Human SMC- and VIC-derived EV abundance and size in conditioned media from cells cultured in control NM or OM with control siRNA (control) or ANXA1 siRNA (ANXA1si); n=3 donors, data are mean±s.d. (FIG. 7B) Illustration showing experiment design to confirm EV surface localization of ANXA1. ANXA1 western blots from human SMC- and VIC-derived EVs treated with EDTA to release ANXA1 on the EV surface into the supernatant; n=3 donors, data are mean±s.d., *P<0.05, analyzed by t-test. (FIG. 7C) DRP1 and β-actin (loading control) protein from SMCs cultured in NM or OM with control siRNA or ANXA1 siRNA; n=3 donors, data are mean±s.d., **P<0.01, analyzed by ANOVA. (FIG. 7D) ANXA1 and flotillin-1 (loading control) protein on EVs derived from SMCs in NM, OM, or OM treated with mdivi-1; n=3 donors, data are mean±s.d., *P<0.05, analyzed by ANOVA.

FIGS. 8A-8D demonstrate that ANXA1 tethered vesicles and its inhibition suppressed vesicle aggregation. (FIG. 8A) Confocal Z stack image of DiR; DiIC18(7) 1,1¹-Dioctadecyl-3,3,3′,3′-Tetramethylindotricarbocyanine Iodide-labeled swelling generated vesicle (X, Y planes, 30 μm, and Z, 10 μm) and ANXA1-GFP binding at tethered sites (arrow) of aggregated vesicles (X, Y planes, 32.4559 μm, and Z, 19.2 μm); n=3 experiments, with representative images shown. (FIG. 8B) ANXA1 time- and concentration-dependent vesicle aggregation; data are mean±s.d. from 3 experiments. (FIG. 8C) EDTA and (FIG. 8D) N-terminal ANXA1 neutralizing antibody vesicle aggregation inhibition. Data are mean±s.d. from 3 experiments; *** *P<0.0001, analyzed by ANOVA.

FIGS. 9A-9B demonstrate that ANXA1 inhibition suppressed SMC- and VIC-derived EV-generated microcalcification in 3D-collagen hydrogels. (FIG. 9A) Illustration showing 3D-collagen hydrogel EV microcalcification experiment design. EV-generated microcalcification in 3D-collagen hydrogels using human SMC- and VIC-derived EVs in conditioned control NM or OM with control siRNA (control) or ANXA1 siRNA (ANXA1si); scale bars, 20 μm, n=3 donors, ***P<0.001, *P <0.05, analyzed by ANOVA. (FIG. 9B) Super resolution imaging of OM SMC- and VIC-derived EV calcification aggregates in 3D-collagen hydrogels. SMC-derived EVs X, Y planes, 3200 nm, Z, 800 nm, and VIC-derived EVs X, 10800 nm, Y, 9600, Z, 4800; n=3 donors, with representative images shown.

FIG. 10 depicts calcified human carotid artery tissue transmission electron microscopy of ANXA1 immunogold labeling (black dots) on tethered EVs (arrows) in collagen extracellular matrix. Scale bars, 100 nm; n=3 donors, with representative images shown, and additional image included in FIG. 5C.

FIG. 11 dsepicts calcified human aortic valve tissue transmission electron microscopy of ANXA1 immunogold labeling (black dots) on tethered EVs (arrows) in collagen extracellular matrix. Scale bars, 100 nm; n=3 donors, with representative images shown, and additional image included in FIG. 5C.

FIGS. 12A-12B depict ANXA1 immunohistochemistry in non-calcified and calcified human carotid artery (FIG. 12A) and aortic valve tissues (FIG. 12). Scale bars, 50 μm; n=5 donors, with 3 donors shown and additional images included in FIG. 5A.

FIG. 13 depicts graphs of unlabeled proteomics analysis of annexins A1, A2, A5, A6, and S100A11 in non-calcified and calcified human aortic valve tissue; n=9 donors, with S100A11 detected in 8/9 non-calcified donor tissues and 7/9 calcified donor tissues, ANXA1 detected in 9/9 donors, ANXA2 detected in 9/9 donors, ANXAS detected in 9/9 donors, and ANXA6 detected in 5/9 non-calcified donor tissues and 1/9 calcified tissues.

FIGS. 14A-14B demonstrate that ANXA1 siRNA did not alter ANXA2, ANX45, and ANXA6 mRNA levels. (FIG. 14A) Human SMCs and (FIG. 14B) VICs ANXA2, ANX45, and ANXA6 mRNA levels from cells cultured in control NM or OM and treated with control siRNA (control) or ANXA1 siRNA (ANXA1si). Data are mean±s.d. from 3 donors; *P<0.05, analyzed by ANOVA.

FIGS. 15A-15B demonstrate immunopurification of GFP-ANXA1 protein and extrusion generated vesicles nanoparticle tracking analysis. (FIG. 15A) Live cell confocal microscopy of HEK-293 cells transfected with human ANXA1-green fluorescent protein (GFP) tagged plasmid; scale bars, 50 μm. Immunoprecipitation western blot of eluted ANXA1-GFP protein used for confocal imaging of ANXA1 binding to polyvinyl alcohol swelling generated phosphatidylserine vesicles. (FIG. 15B) Nanoparticle tracking analysis of filter extrusion generated phosphatidylserine vesicles size; data are mean±s.d. from 5 replicate measurements.

FIG. 16 depicts a diagram of a working model: In human SMCs or VICs calcifying conditions altered mitochondrial calcium storage and calcium signaling that increased TNAP activation via annexin calcium channels. TNAP was then loaded in EVs, increasing EV calcification potential. Calcium signaling alterations led to annexin localization on cell membrane leading to increased annexin, including ANXA1 and annexin-associated proteins (S100 calcium binding proteins) incorporation on EVs that promoted tethering and aggregation of EVs trapped in collagen extracellular matrix. Aggregated EVs then nucleated hydroxyapatite and formed microcalcifications. ANXA1 inhibition suppressed TNAP activity and EV tethering, thereby attenuating microcalcification formation.

FIG. 17 depicts a diagram of a working model. Osteogenic conditions lead to TNAP activation and loading (as shown in Goettsch et al. 2016) into extracellular vesicles (EV), and altered calcium signaling (as shown in Rogers et al. 2017) as that leads to annexin trafficking changes and increased annexin on the surface of EVs, which in turn enhances EV tethering promoting the formation of microcalcifications (as shown in Hutcheson et al. 2016). Annexin al inhibition reduces calcification formation by inhibiting TNAP activation and vesicle tethering.

FIG. 18 depicts exemplary scanning electron microscopy images of calcification in human artery and valve. For image clarification, the artery image has been colored by using density-dependent coloration in which collagen and microcalcifications appear. These images shown that vesicles can contribute to tissue calcification and are aggregated in human tissue. Extracellular vesicles tether in calcified human carotid artery and valve tissue. Representative electron microscopy images showing aggregated vesicles in collagen extracellular matrix in calcified human carotid artery and calcified aortic valve tissue; n=3. Annexin A1 is increased in osteogenic media cultured human smooth muscle cell vesicles. Volcano plot of secreted proteins in human coronary artery smooth muscle cell extracellular vesicles cultured in control and osteogenic media; osteogenic media ratio compared to control media shown. Arrow depicts annexin al, which is significantly enriched in osteogenic media vesicles; n=9.

FIG. 19 demonstrates that calcified human artery and valve tissue vesicles contain annexin al. Representative IHC and positive annexin A1 staining in human cardiovascular tissue (secondary only used as control), and immunogold electron microscopy images showing aggregated vesicles labeled with annexin al immunogold in collagen extracellular matrix in calcified human aortic valve and artery tissue; n=3-5.

FIG. 20 demonstrates that Annexin A1 siRNA reduced human smooth muscle cell and valve interstitial cell tissue non-specific alkaline phosphatase activity. Human smooth muscle cell and valve interstitial cell siRNA knockdown confirmation by western blot and qPCR, along with confirmation that annexin al siRNA did not alter other annexin mRNA levels; NM=control media, OM=osteogenic media. Human coronary artery smooth muscle cell and valve interstitial cell tissue non-specific alkaline phosphatase activity was measured at day 14; n=3.

FIG. 21 demonstrates that Annexin A1 siRNA reduced human smooth muscle cell calcification. Annexin al siRNA did not alter extracellular vesicle (EV) abundance or size. Annexin al siRNA suppressed human smooth muscle cell vesicle mediated calcification in 3D collagen hydrogels. Confocal imaging shown of all conditions and super resolution shown for the osteogenic media condition microcalcifications. 3D collagen hydrogen model of vesicle mediated calcification, showing Annexin A1 siRNA attenuation of calcification. NM=control media, OM=osteogenic media, Scr siRNA=control siRNA, annexin al siRNA=annexin al knockdown siRNA. Day 14 human coronary artery smooth muscle cell media containing extracellular vesicles was added to cell-free 3D collagen hydrogels and then cultured for 21 days at which point collagen was stained by CNA collagen probe and calcification with Osteosense; n=3.

FIG. 22 demonsrates that Annexin A1 siRNA reduced human valve interstitial calcification. Annexin al siRNA did not alter extracellular vesicle (EV) abundance or size. Annexin al siRNA suppressed human valve interstitial cell vesicle mediated calcification in 3D collagen hydrogels. Confocal imaging shown of all conditions and super resolution shown for the osteogenic media condition microcalcifications. 3D collagen hydrogen model of vesicle mediated calcification, showing Annexin A1 siRNA attenuation of calcification. NM=control media, OM=osteogenic media, Scr siRNA=control siRNA, annexin al siRNA=annexin al knockdown siRNA. Day 14 human valve interstitial cell media containing extracellular vesicles was added to cell-free 3D collagen hydrogels and then cultured for 21 days at which point collagen was stained by CNA collagen probe and calcification with Osteosense; n=3.

### DETAILED DESCRIPTION

As described herein, the inventors have found that ANXA1 contributes to vesicle aggregation and microcalcification formation. Accordingly, provided herein are methods of treating or preventing calcification, microcalcification, and various vascular/valvular/cardiovascular conditions by inhibiting ANXA1, thereby preventing the mechanism of clacification.

Notably, the current findings demonstrate that the processes of atherosclerosis lesion and calcification formation are different mechanistically. Where ANXA1 itself may be therapeutic in murine atherosclerosis lesions, in human calcification it is the inhibition of ANXA1 that is therapeutic. It is contemplated herein that this effect of ANXA1 on murine atherosclerosis lesions is due to influencing neutrophil activity. Such an effect is unique to mice, as there is no evidence of neutrophil involvement in human atherosclerosis. In some embodiments of any of the aspects, the subject treated according to the present methods is a human subject. In some embodiments of any of the aspects, the subject treated according to the present methods does not have or is not diagnosed with atherosclerosis and/or atherosclerotic lesions. In some embodiments of any of the aspects, the subject treated according to the present methods does not have or is not diagnosed with late stage atherosclerosis and/or atherosclerotic lesions.

In one aspect of any of the embodiments, described herein is a method of treating an extracellular vesicle (EV)-associated disease in subject, the method comprising: administering an agent that reduces the levels or activity of Annexin A1 (ANXA1) in a subject in need thereof In one aspect of any of the embodiments, described herein is a method of reducing vascular calcification in subject, the method comprising administering an agent that reduces the levels or activity of Annexin Al (ANXA1) in a subject in need thereof. In one aspect of any of the embodiments, described herein is a method of treating, reducing, or preventing microcalcification/calcification in subject, the method comprising administering an agent that reduces the levels or activity of Annexin A1 (ANXA1) in a subject in need thereof In one aspect of any of the embodiments, described herein is a method of treating, reducing, or preventing plaque rupture in subject, the method comprising administering an agent that reduces the levels or activity of Annexin A1 (ANXA1) in a subject in need thereof

As used herein, the term “ extracellular vesicle ” refers to a cell-derived vesicle comprising a membrane surrounding the inner space. The term extracellular vesicles comprises all membrane-bound vesicles having a smaller diameter than the cells from which they are derived, including but not limited to exosomes, microparticles and shed microvesicles secreted under either normal physiological and pathological conditions. Typically, the extracellular vesicles are in the range of 20 nm to 1000 nm in diameter

As used herein “annexin A1” or “ANXA1” refers to membrane-associated phospholipid-bindig protein. ANXA1 sequences are known in the art for a number of species, e.g., human ANXA1 (NCBI Gene ID: 301) mRNA (e.g., NCBI Ref Seq: NM_000700.3; SEQ ID NO: 4) and polypeptide sequenes (e.g. NCBI Ref Seq: NP_000691.1; SEQ ID NO: 3).

An agent that reduces the levels of activity of Annexin A1 (ANXA1) can be any agent that reduces the level or activity of ANXA1, either by interacting directly (i.e. physically) with an ANXA1 gene or gene product (direct inhibition) or by acting directly on another target that then interacts with an ANXA1 gene or gene product (indirect inhibition). The level of ANXA1 can be the level of ANXA1 in the cell, the level of ANXA1 in a subject or in a sample obtained from a subject, and/or the level of ANXA1 in EVs. The activity of ANXA1 can the ability of ANXA1 to cause EV aggregation, the presence of ANXA1 on EV surfaces, and/or the ability of ANXA1 to induce calcification/microcalcification. In some embodiments of any of the aspects, the agent that reduces the levels of activity of ANXA1 and/or the inhibitor of ANXA1 is specific for ANXA1, e.g., it does not inhibit ANXA3, ANXA4, ANXAA11, ANXAA2, ANXA5, and/or ANXA6.

The terms “compound” and “agent” refer to any entity which is normally not present or not present at the levels being administered and/or provided to a cell, tissue or subject. An agent can be selected from a group comprising: chemicals; small organic or inorganic molecules; signaling molecules; nucleic acid sequences; nucleic acid analogues; proteins; peptides; enzymes; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments of any of the aspects, the agent is any chemical, entity or moiety, including without limitation synthetic and naturally occurring non-proteinaceous entities. In certain embodiments the agent is a small molecule having a chemical moiety. For example, chemical moieties include unsubstituted or substituted alkyl, aromatic, or heterocyclyl moieties including macrolides, leptomycins and related natural products or analogues thereof. Agents can be known to have a desired activity and/or property or can be selected from a library of diverse compounds.

In some embodiments of any of the aspects, the agent that reduces the levels or activity of Annexin A1 (ANXA1) is an inhibitor of ANXA1, e.g., a direct inhibitor as described above herein. As used herein, the term “inhibitor” refers to an agent which can decrease the expression and/or activity of the targeted expression product (e.g. mRNA encoding the target, or a target polypeptide), e.g. by at least 10% or more, e.g. by 10% or more, 50% or more, 70% or more, 80% or more, 90% or more, 95% or more, or 98% or more. The efficacy of an inhibitor of, for example, ANXA1, e.g. its ability to decrease the level and/or activity of ANXA1 can be determined, e.g. by measuring the level of ANXA1 protein (or its mRNA). Methods for measuring the level of a given mRNA and/or polypeptide are known to one of skill in the art, e.g. RT-PCR with primers can be used to determine the level of RNA, and Western blotting with an antibody can be used to determine the level of a polypeptide. In some embodiments of any of the aspects, an inhibitor can be an inhibitory nucleic acid; an aptamer; an antibody reagent; an antibody; or a small molecule. Exemplary inhibitors, e.g., of ANXA1 can include a small molecule, nucleic acid (e.g., inhibitory nucleic acid), polypeptide, antibody reagent, or genome editing system.

In some embodiments of any of the aspects, the agent that inhibits, e.g. ANXA1, is an inhibitory nucleic acid. In some embodiments of any of the aspects, inhibitors of the expression of a given gene can be an inhibitory nucleic acid. As used herein, “inhibitory nucleic acid” refers to a nucleic acid molecule which can inhibit the expression of a target, e.g., double-stranded RNAs (dsRNAs), inhibitory RNAs (iRNAs), and the like. In some embodiments of any of the aspects, the inhibitory nucleic acid can be a silencing RNA (siRNA), microRNA (miRNA), or short hairpin RNA (shRNA).

In some embodiments of any of the aspects, the iRNA can comprise, consist of, or consist essentially of the sequence of SEQ ID NO: 1 (ANXA1 siRNA (Fisher Scientific, #4390824); NCBI Ref Seq: NM_00245.3) or SEQ ID NO: 2 (ANXA1 siRNA (Fisher Scientific, #4390825); NCBI Ref Seq: NM_000700.3). One skilled in the art would be able to design further iRNA, siRNA, shRNA, or miRNA to target the nucleic acid sequence of ANXA1 (e.g., SEQ ID NO: 4), e.g., using publically available design tools. iRNA, siRNA, shRNA, or miRNA is commonly made using companies such as Dharmacon (Layfayette, Colo.) or Sigma Aldrich (St. Louis, Mo.).

In some embodiments of any of the aspects, ANXA1 is depleted from a genome, using any genome editing system including, but not limited to, zinc finger nucleases, TALENS, meganucleases, and CRISPR/Cas systems. In some embodiments of any of the aspects, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In some embodiments of any of the aspects, the inhibitor of ANXA1 can be an anti-ANXA1 antibody reagent, e.g., an antibody reagent that binds specifically to ANXA1. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

In some embodiments of any of the aspects, the antibody reagent can be an ANXA1-neutralizing antibody reagent, e.g., an antibody that inhibits or reduces the ability of ANXA1 to tether EVs. In some embodiments of any of the aspects, a ANXA1-neutralizing antibody reagent is one that binds to the N-terminal domain of ANXA1, which is described elsewhere herein. In some embodiments of any of the aspects, a ANXA1-neutralizing antibody reagent is one that binds to some or all of residues 1-46 of ANXA1 (e.g., residues 1-46 of SEQ ID NO: 3).

Antibody reagents as described herein, e.g., anti-ANXA1 or ANXA1-neutralizing antibodies are known in the art. For example, such reagents are readily commercially available. In some embodiments of any of the aspects, an antibody reagent specific for a target described herein (e.g., that binds specifically to the target) can be an antibody reagent comprising one or more (e.g., one, two, three, four, five, or six) CDRs of any one of the antibodies recited in Table 1. In some embodiments of any of the aspects, an antibody reagent specific for a target and/or maker described herein can be an antibody reagent comprising the six CDRs of any one of the antibodies recited in Table 1. In some embodiments of any of the aspects, an antibody reagent specific for a target described herein can be an antibody reagent comprising the three heavy chain CDRs of any one of the antibodies recited in Table 1. In some embodiments of any of the aspects, an antibody reagent specific for a target described can be an antibody reagent comprising the three light chain CDRs of any one of the antibodies recited in Table 1. In some embodiments of any of the aspects, an antibody reagent specific for a target described herein can be an antibody reagent comprising the VH and/or VL domains of any one of the antibodies recited in Table 1. In some embodiments of any of the aspects, an antibody reagent specific for a target described herein can be an antibody reagent comprising the VH and VL domains of any one of the antibodies recited in Table 1. In some embodiments of any of the aspects, an antibody reagent specific for a target described herein can be an antibody reagent recited in Table 1. Such antibody reagents are specifically contemplated for use in the methods and/or compositions described herein.

TABLE 1

| Antibody Clone | |
|---|---|
| Designation | Source |
| 7H46L26 | Cat. No. 702057; ThermoFisher (Waltham, MA) |
| OTI3A8 | Cat. No. CF500967; ThermoFisher (Walthham, MA) |
| 1E1B7 | Cat. No. 66344-1-Ig; Proteintech (Rosemount, IL) |
| CL0199 | Cat. No. AMAb90558; Atlas Antibodies (Stockholm, Sweden) |
| CPTC-ANXA1-2 | Cat. No. MABS45; EMD Millipore (Burlington, MA) |
| PAT2G5AT | Cat. No. ANT-562; ProSpec (Ness-Ziona, Israel) |
| | Cat. No. Ab86446; Abcam (Cambridge, UK) |
| | (The immunogen used to make this antibody was a peptide corresponding |
| | to residues 1-50 of human ANXA1) |
| | Cat. No. Ab65844; Abeam (Cambridge, UK) |
| | (The immunogen used to make this antibody was a peptide corresponding |
| | to residues 3-24 of human ANXA1) |
| | Cat. No. Ab33061; Abcam (Cambridge, UK) |
| | (The immunogen used to make this antibody was a peptide corresponding |
| | to residues 1-50 of human ANXA1) |
| | LS-C136979-100; LifeSpan BioSciences (Seattle, WA) |
| | (The immunogen used to make this antibody was a peptide corresponding |
| | to the N-terminus of ANXA1) |
| | LS-C358901-30; LifeSpan BioSciences (Seattle, WA) |
| | (The immunogen used to make this antibody was a peptide corresponding |
| | to the N-terminus of ANXA1) |
| | 119-13624; RayBiotech (Peachtree Corners, GA) |
| | (The immunogen used to make this antibody was a peptide corresponding |
| | to the N-terminus of ANXA1) |
| | LS-B3363-250; LifeSpan BioSciences (Seattle, WA) |
| | (The immunogen used to make this antibody was a peptide corresponding |
| | to the N-terminus of ANXA1) |
| EPR19342 | Cat. No. ab214486; Abeam (Cambridge, UK) |
| 3A8 | LS-C115019-100; LifeSpan BioSciences (Seattle, WA) |
| 5E4/1 | Orb223639; Biorbyt (Cambridge, UK) |
| 5E4-D8-F12 | MBS475176; MyBioSource (San Diego, CA) |
| MRQ-3 | MBS370183; MyBioSource (San Diego, CA) |
| 74/3 | 831601; BioLegend (San Diego, CA) |
| M021 | AM0211; ECM Biosciences (Versailles, KY) |
| M018 | AM0181; ECM Biosciences (Versailles, KY) |
| ANEX 6E4-3 | ABIN5621667; antibodies-online (Limerick, PA) |
| CPTC22 | VMA00558; Bio-Rad (Hercules, CA) |
| | LS-C393678-100; LifeSpan Biosciences (Seattle, WA) |
| | LS-C306262-100; Life Span Biosciences (Seattle, WA) |
| IHC512 | GTX57177; GeneTex (Irvine, CA) |
| 2F1 | Ab135256; Abcam (Cambridge, UK) |
| | ITM1434-100u; G Biosciences |
| 17J8 | 381568-100ul; United States Biological (Salem, MA) |
| AT2G5 | IBATGA0336; Immuno-Biological Laboratories (Minneapolis, MN) |
| 39 | Sc-130305; Santa Cruz Biotechnology (Dallas, TX) |
| EH17a | Sc-12740; Santa Cruz Biotechnology (Dallas, TX) |
| | 101-10082; RayBiotech (Peachtree Corners, GA) |
| SAIC-13B-19 | AB00306-1.1; Absolute Antibody (Boston, MA) |
| 4C1 | Bsm-51219M; Bioss Inc. (Woburn, MA) |
| 3G2 | DCABH-3214; Creative Diagnostics (Shirley, NY) |
| 4B9 | DCABH-675; Creative Diagnostics (Shirley, NY) |

In some embodiments of any of the aspects, an antibody reagent specific for a target described herein (e.g., that binds specifically to the target) can be an antibody reagent comprising one or more (e.g., one, two, three, four, five, or six) CDRs of any one of the antibodies recited in Table 2. In some embodiments of any of the aspects, an antibody reagent specific for a target and/or maker described herein can be an antibody reagent comprising the six CDRs of any one of the antibodies recited in Table 2. In some embodiments of any of the aspects, an antibody reagent specific for a target described herein can be an antibody reagent comprising the three heavy chain CDRs of any one of the antibodies recited in Table 2. In some embodiments of any of the aspects, an antibody reagent specific for a target described can be an antibody reagent comprising the three light chain CDRs of any one of the antibodies recited in Table 2. In some embodiments of any of the aspects, an antibody reagent specific for a target described herein can be an antibody reagent comprising the VH and/or VL domains of any one of the antibodies recited in Table 2. In some embodiments of any of the aspects, an antibody reagent specific for a target described herein can be an antibody reagent comprising the VH and VL domains of any one of the antibodies recited in Table 2. In some embodiments of any of the aspects, an antibody reagent specific for a target described herein can be an antibody reagent recited in Table 2. Such antibody reagents are specifically contemplated for use in the methods and/or compositions described herein.

TABLE 2

| Antibody Clone | |
|---|---|
| Designation | Source |
| | Cat. No. Ab86446; Abcam (Cambridge, UK) |
| | (The immunogen used to make this antibody was a peptide corresponding |
| | to residues 1-50 of human ANXA1) |
| | Cat. No. Ab65844; Abcam (Cambridge, UK) |
| | (The immunogen used to make this antibody was a peptide corresponding |
| | to residues 3-24 of human ANXA1) |
| | LS-C136979-100; LifeSpan BioSciences (Seattle, WA) |
| | (The immunogen used to make this antibody was a peptide corresponding |
| | to the N-terminus of ANXA1) |
| | LS-C358901-30; LifeSpan BioSciences (Seattle, WA) |
| | (The immunogen used to make this antibody was a peptide corresponding |
| | to the N-terminus of ANXA1) |
| | 119-13624; RayBiotech (Peachtree Corners, GA) |
| | (The immunogen used to make this antibody was a peptide corresponding |
| | to the N-terminus of ANXA1) |
| | LS-B3363-250; LifeSpan BioSciences (Seattle, WA) |
| | (The immunogen used to make this antibody was a peptide corresponding |
| | to the N-terminus of ANXA1) |

In some embodiments of any of the aspects, an inhibitor of ANXA1 can be an agent that cleaves (or induces cleavage of) ANXA1 such that some or all of the N-terminal domain of ANXA1 is separated from the the rest of the protein. As used herein “N-terminal domain” of ANXA1 refers to the portion of ANXA1 which is N-terminal to the annexin domains, e.g., residues 1-46 of SEQ ID NO:3. In some embodiments of any of the aspects, the N-terminal domain of ANXA1 refers to residues 1-11, 1-22, or 1-36 of SEQ ID NO:3. In some embodiments of any of the aspects, cleavage of ANXA1 can comprise separation of at least residues 1-11 of SEQ ID NO:3 from the rest of the protein. In some embodiments of any of the aspects, cleavage of ANXA1 can comprise separation of at least residues 1-22 of SEQ ID NO:3 from the rest of the protein. In some embodiments of any of the aspects, cleavage of ANXA1 can comprise separation of at least residues 1-36 of SEQ ID NO:3 from the rest of the protein. Cleavage of one or more of these sequences can be peformed by proteinase 3 and/or HLE (see, e.g., Vong et al. J Biol Chem 282:29998-30004 (2007) and Rescher et al. BBA 1763:1320-4 (2006); each of which is incorporated by reference herein in its entirety). Accordingly, in some embodiments, the agent that induces cleavage of the N-terminal domain of ANXA1 can be an agonist of proteinase 3 or HLE.

As used herein “neutrophil expresse elastase”, “HLE”, or “ELANE” refers to a protease that targets elastin and ANXA1. HLE sequences are known in the art for a number of species, e.g., human HLE (NCBI Gene ID: 1991) mRNA (e.g., NCBI Ref Seq: NM_001972.4; SEQ ID NO: 5) and polypeptide sequences (e.g. NCBI Ref Seq: NP_001963.1; SEQ ID NO: 6).

As used herein “proteinase 3” or “PRTN3” refers to a protease that targets ANXA1. PRTN3 sequences are known in the art for a number of species, e.g., human PRTN3 (NCBI Gene ID: 5657) mRNA (e.g., NCBI Ref Seq: NM_002777.4; SEQ ID NO: 7) and polypeptide sequences (e.g. NCBI Ref Seq: NP_002768.3; SEQ ID NO: 8).

As used herein, the term “agonist” refers to an agent which increases the expression and/or activity of the target by at least 10% or more, e.g. by 10% or more, 50% or more, 100% or more, 200% or more, 500% or more, or 1000% or more. The efficacy of an agonist of, for example, HLE or proteinase 3, e.g. its ability to increase the level and/or activity of the target can be determined, e.g. by measuring the level of an expression product of the target and/or the activity of target. Methods for measuring the level of a given mRNA and/or polypeptide are known to one of skill in the art, e.g. RTPCR with primers can be used to determine the level of RNA, and Western blotting with an antibody can be used to determine the level of a polypeptide. Suitable primers for a given target are readily identified by one of skill in the art, e.g., using software widely available for this purpose (e.g., Primer3 or PrimerBank, which are both available on the world wide web). Antibodies to proteinase 3 and HLE are commercially available. Assays for measuring the activity of the targets, e.g. the level of cleavage of the N-terminal domain of ANXA1 are known in the art (see, e.g., Vong et al. J Biol Chem 282:29998-30004 (2007) and Rescher et al. BBA 1763:1320-4 (2006); each of which is incorporated by reference herein in its entirety). Non-limiting examples of agonists of a given polypeptide target can include the target polypeptides or variants or functional fragments thereof and nucleic acids encoding the polypeptide or variants or functional fragments thereof In some embodiments of any of the aspects, the agonist of proteinase 3 is a proteinase 3 polypeptide or variants or functional fragment thereof and/or a nucleic acid encoding the polypeptide or variant or functional fragment thereof. In some embodiments of any of the aspects, the agonist of HLE, is a HLE polypeptide or variants or functional fragment thereof and/or a nucleic acid encoding the polypeptide or variant or functional fragment thereof. In some embodiments of any of the aspects, the agonist of, e.g. HLE or PRTN3 can be a HLE or PRTN3 polypeptide. In some embodiments of any of the aspects, the polypeptide agonist can be an engineered and/or recombinant polypeptide. In some embodiments of any of the aspects, the polypeptide agonist can be a nucleic acid encoding a polypeptide, e.g. a functional fragment thereof. In some embodiments of any of the aspects, the nucleic acid can be comprised by a vector.

In some embodiments of any of the aspects, a HLE or PRTN3 agonist can be a polypeptide comprising the sequence of a human HLE or PRTN3 polypeptide, e.g., SEQ ID NO: 6 or SEQ ID NO: 8. In some embodiments of any of the aspects, a HLE or PRTN3 agonist can be a polypeptide consisting essentially of the sequence of a human HLE or PRTN3 polypeptide, e.g., SEQ ID NO: 6 or SEQ ID NO: 8. In some embodiments of any of the aspects, a HLE or PRTN3 agonist can be a polypeptide consisting of the sequence of a human HLE or PRTN3 polypeptide, e.g., SEQ ID NO: 6 or SEQ ID NO: 8. In some embodiments of any of the aspects, an HLE or PRTN3 agonist can be a polypeptide comprising the reference sequence with at least 80%, at least 85%, at least 90%, at least 95%, or at least 98% identity to one of SEQ ID NOs: 6 or 8 and which retains the ANXA1-cleaving activity of a polypeptide of one of SEQ ID NOs: 6 or 8. In some embodiments of any of the aspects, an agonist can be a nucleic acid comprising a sequence which encodes one of the foregoing HLE or PRTN3 polypeptides.

In some embodiments of any of the aspects, the agent that reduces the levels or activity of ANXA1 can be a calcium chelator. Non-limiting examples of calcium chelators include one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), DM-NITROPHENTM, and pharmaceutically acceptable salts thereof. In some embodiments of any of the aspects, the calcium chelator is ethylenediaminetetraacetic acid (EDTA).

An agent can reduce the levels or activity of ANXA1 by reducing the rate or amount of ANXA1 loaded into extraceullar vesicles. As described herein, this process is controlled at least in part by calcium levels in the cell from which the EV originates. Increasing mitochondrial calcium storage by inhibiting DRP1-mediated mitochondrial fission leading to reduced loading of ANXA1 on extracellular vesicles via suppression of cytosolic calcium-driven ANXA1 trafficking. DRP1 inhibition increases mitochondrial calcium storage, thereby reducing calcium release into the cell cytosol. Mitochondrial fission-mediated release of free calcium to the cytosol leads to calcium binding cytosolic ANXA 1. Binding of calcium to cytosolic ANXA1 and related calcium binding proteins leads to increased incorporation of these proteins on the surface of extracellular vesicles, where they induce vesicle aggregation and microcalcification formation. Accordingly, increasing the levels of mitrochondrial calcium, increasing mitrochondrial calcium storage, and/or decreasing cytosolic calcium inhibits the loading of ANXA1 into the EVs. Such a change in calcium levels can be achieved by, e.g., an inhibitor of dynamin-related protein 1 (DRP1 or DNM1L) (e.g., NCBI Gene ID: 10059; exemplary polypeptides (NP_001265392.1, NP_{—} 001265393.1, NP_{—} 001265394.1, NP_001265395.1, NP_001317309.1, NP_005681.2, NP_036192.2, NP_036193.2 (SEQ ID NOs: 9-16, respectively)) and mRNAs (NM_001278463.1, NM_{—} 001278464.1, NM_{—} 001278465.1, NM_001278466.1, NM_001330380.1, NM_005690.4, NM_012062.5, and NM_012063.3 (SEQ ID NOs: 17-24, respectively)) . Suitable types of inhibitors for a given target gene/protein are described elsewhere herein and apply equally to direct inhibitors of DRP1. For example, an inhibitors of DRP1 can be an inhibitory nucleic acid, antibody reagent, and/or small molecule. In some embodiments of any of the aspects, the inhibitor dynamin-related protein 1 (DRP1) is an inhibitory nucleic acid, e.g., a siRNA, or is mdivi-1 (a compound of Formula I).

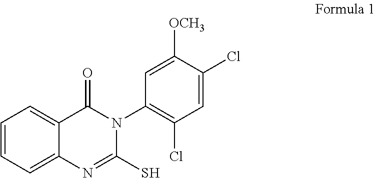 [SMILES: COc1cc(-n2c(S)nc3ccccc3c2=O)c(Cl)cc1Cl]

In some embodiments of any of the aspects, multiple agents can be administered to the subject or cell, e.g., multiple agents that reduce the levels or activity of ANXA1, an indirect and a direct inhibitor of ANXA1, or multiple types of direct inhibitors of ANXA1. Any combination of the types and individual agents described herein are contemplated for use in the presently described methods. In aspects that relate to compositions, a composition described herein can likewise comprise a combination of any two or more types and/or individual agents described herein. Such a combination can be a mixture (e.g., a single formulation), or a kit or packaging comprising multiple formulations to be mixed or administered/used as separate formulations.

As described, EVs can promote, cause, or contribute to a number of diseases, e.g., by aggregrating in the vasculature and/or inducing calcification. Diseases and conditions which are caused by or associated with aberrant EV production and/or aggregration are referred to herein as “extracellular vesicle (EV)-associated diseases.” Non-limiting examples of EV-associated diseases include valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity. In some embodiments of any of the aspects, the subject treated according to the methods described herein has or is in need of treatment for a condition selected from valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.

In one aspect of any of the embodiments, described herein is a method for inhibiting or reducing aggregation of extracellular vesicles (EV) in a subject or system the method comprising: contacting the system with or administering to the subject an agent that reduces the levels or activity of ANXA1. In one aspect of any of the embodiments, described herein is a method for inhibiting or reducing trafficking of an extracellular vesicle (EV) from a cell, the method comprising: contacting the cell with an agent that reduces the levels or activity of ANXA1. Inhibiting or reducing trafficking can comprise a decrease in the rate or number of EV's leaving a cell or population of cells. The cell can be ex vivo, in vivo, or in vitro. In some embodiments of any of the aspects, the cell is a smooth muscle cell (SMC), a valvular interstitial cell (VIC), oligodendroglioma cell, endothelial cell, macrophage, monocyte, or cancer cell.

In some embodiments, the methods described herein relate to treating a subject having or diagnosed as having calcification (e.g. in the cardiovascular system) with one or more agents described herein. Subjects having calcification (e.g., in a tissue other than bone) can be identified by a physician using current methods of diagnosing calcification. Symptoms and/or complications of calcification which characterize these conditions and aid in diagnosis are well known in the art and include but are not limited to, impair heart or circulatory function. Tests that may aid in a diagnosis of, e.g. calcification include, but are not limited to, radiography and/or ultrasounds. A family history of calcification, or exposure to risk factors for calcification (e.g. smoking is dyslipidemia) can also aid in determining if a subject is likely to have calcification or in making a diagnosis of calcification.

As explained herein, agents which reduce the levels or activity of ANXA1 are particularly effective in reducing or preventing calcifications or microcalcifications. Accordingly, in some embodiments of any of the aspects, a subject treated according to the methods described herein, e.g., having or diagnosed as having an EV-associated disease, can be a subject with calcifications or microcalcifications in addition to the diagnosis of an EV-associated disease. In some embodiments of any of the aspects, the method described heren further comprises receiving the results of an assay that indicates that the subject has an increase in the level of microcalcifications or calcifications in the subject before administering the agent that reduces the levels or activity of ANXA1. In some embodiments of any of the aspects, the method described heren further comprises a first step of performing a test or assay to determine the level of microcalcifications or calcifications in the subject and administering an agent that reduces the levels or activity of ANXA1 if an increased level is detected. In some embodiments of any of the aspects, the method described heren further comprises a first step of performing a test or assay to determine the level of microcalcifications or calcifications in the subject and administering an agent that reduces the levels or activity of ANXA1 if an increased level is detected or administering an agent for treating the EV-associated disease which does not reduce the level or activity of ANXA1 if an increased level is not detected (e.g., a statin or anti-inflammatory). In some embodiments of any of the aspects, the method described heren further comprises a first step of determining the level of microcalcifications or calcifications in the subject and administering agent that reduces the levels or activity of ANXA1 if an increased level is detected or administering an agent for treating the EV-associated disease which does not reduce the level or activity of ANXA1 if an increased level is not detected (e.g., a statin or anti-inflammatory).

In some embodiments of any of the aspects, the method comprises administering an agent that reduces the levels or activity of ANXA1 to a subject previously determined to have a level of calcification/microcalcifications that is increased relative to a reference. In some embodiments of any of the aspects, described herein is a method of treating an EV-associated diease in a subject in need thereof, the method comprising: a) first determining the level of calcifications/microcalcifications in the subject; and b) then administering an agent that reduces the levels or activity of ANXA1 to the subject if the level of calcifications/microcalcifications is increased relative to a reference. In some embodiments of any of the aspects, the step of determining if the subject has an increased level of calcifications/microcalcifications can comprise i) optionally obtaining or having obtained a sample from the subject and ii) performing or having performed an assay/test on the sample obtained from the subject or the subject to determine/measure the level of calcifications/microcalcifications in the subject. In some embodiments of any of the aspects, the step of determining if the subject has an increased level of calcifications/microcalcifications can comprise performing or having performed an assay/test on a sample obtained from the subject or the subject to determine/measure the level of calcifications/microcalcifications in the subject. In some embodiments of any of the aspects, the step of determining if the subject has an increased level of calcifications/microcalcifications can comprise ordering or requesting an assay/test on a sample obtained from the subject or the subject to determine/measure the level of calcifications/microcalcifications in the subject. In some embodiments of any of the aspects, the step of determining if the subject has an increased level of calcifications/microcalcifications can comprise receiving the results of an assay/test on a sample obtained from the subject or the subject to determine/measure the level of calcifications/microcalcifications in the subject. In some embodiments of any of the aspects, the step of determining if the subject has an increased level of calcifications/microcalcifications can comprise receiving a report, results, or other means of identifying the subject as a subject with an increased level of calcifications/microcalcifications. p In one aspect of any of the embodiments, described herein is a method of treating an EV-associated disease in a subject in need thereof, the method comprising: a) determining if the subject has an increased level of calcifications/microcalcifications; and b) instructing or directing that the subject be administered an agent that reduces the levels or activity of ANXA1 if the level of calcification/microcalfication is increased relative to a reference. In some embodiments of any of the aspects, the step of determining if the subject has an increased level of calcification/microcalcification can comprise i) optionally obtaining or having obtained a sample from the subject and ii) performing or having performed an assay/test on the sample obtained from the subject or the subject to determine/measure the level of calcification/microcalcification in the subject. In some embodiments of any of the aspects, the step of determining if the subject has an increased level of calcification/microcalcification can comprise performing or having performed an assay/test on a sample obtained from the subject or the subject to determine/measure the level of calcification/microcalcification in the subject. In some embodiments of any of the aspects, the step of determining if the subject has an increased level of calcification/microcalcification can comprise ordering or requesting an assay/test on a sample obtained from the subject or the subject to determine/measure the level of calcification/microcaclfication in the subject. In some embodiments of any of the aspects, the step of instructing or directing that the subject be administered a particular treatment can comprise providing a report of the assay/test results. In some embodiments of any of the aspects, the step of instructing or directing that the subject be administered a particular treatment can comprise providing a report of the assay/test results and/or treatment recommendations in view of the assay results.

Suitable assays for determining the level of calcifications/microcalcifications in a subject can include radiography, ultrasound, an extracellular vesicle calcification assay; a vesicle binding assay; a vesicle tethering assay; a tissue non-specific alkaline phosphatase (TNAP) assay; an immunohistochemistry assay; mass spectrometry; proteomics; scanning electron microscopy; and transmission electron microscopy. In some embodiments, the level of calcifications/microcalfications is the level in a biological sample obtained from the subject. Exemplary, non-limiting biological samples include blood, a vascular tissue, or a heart valve tissue.

In some embodiments of any of the aspects, measurement of the level of a target and/or detection of the level or presence of a target, e.g. of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. a mRNA or polypeptide can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for the various targets described herein are commercially available and can be used for the purposes of the invention to measure protein expression levels, e.g. anti- ANXA 1. Alternatively, since the amino acid sequences for the targets described herein are known and publically available at the NCBI website, one of skill in the art can raise their own antibodies against these polypeptides of interest for the purpose of the methods described herein. The amino acid sequences of the polypeptides described herein have been assigned NCBI accession numbers for different species such as human, mouse and rat.

In some embodiments of any of the aspects, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments of any of the aspects, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments of any of the aspects, the immunoassay can be a quantitative or a semi-quantitative immunoassay.

An immunoassay is a biochemical test that measures the concentration of a substance in a biological sample, typically a fluid sample such as blood or serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In some embodiments of any of the aspects, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In some embodiments of any of the aspects, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments of any of the aspects, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U. S. patent applications Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, an adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof The membrane is then washed to remove unbound proteins and proteins with non-specific binding. Detectably labeled enzyme-linked secondary or detection antibodies can then be used to detect and assess the amount of polypeptide in the sample tested. A dot blot immobilizes a protein sample on a defined region of a support, which is thenprobed with antibody and labelled secondary antibody as in Western blotting. The intensity of the signal from the detectable label in either format corresponds to the amount of enzyme present, and therefore the amount of polypeptide. Levels can be quantified, for example by densitometry.

In some embodiments of any of the aspects, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the genes described herein. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNAse protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein have been assigned NCBI accession numbers for different species such as human, mouse and rat. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments of any of the aspects, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments of any of the aspects, a detectable label can be a radiolabel including, but not limited to ³H, ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level in a population of subjects who do not have or are not diagnosed as having, and/or do not exhibit signs or symptoms of an EV-associated disease. In some embodiments of any of the aspects, the reference can also be a level in a control sample, in a control subject, a pooled sample of control individuals, in a pool of control individuals, or a numeric value or range of values based on the same. In some embodiments of any of the aspects, the reference can be the level in a sample obtained from the same subject or the subject at an earlier point in time, e.g., the methods described herein can be used to determine if a subject's sensitivity or response to a given therapy is changing over time.

In some embodiments of any of the aspects, the level of expression products of no more than 200 other genes is determined. In some embodiments of any of the aspects, the level of expression products of no more than 100 other genes is determined. In some embodiments of any of the aspects, the level of expression products of no more than 20 other genes is determined. In some embodiments of any of the aspects, the level of expression products of no more than 10 other genes is determined.

In some embodiments of the foregoing aspects, the expression level of a given gene can be normalized relative to the expression level of one or more reference genes or reference proteins.

In some embodiments, the reference level can be the level in a sample obtained from a subject of similar age, sex and other demographic parameters as the sample/subject for which the level of calcification/microcalcification is to be determined. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject. The test sample can be obtained by removing a sample from a subject, but can also be accomplished by using a previously isolated sample (e.g. isolated at a prior timepoint and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having) an EV-associated disease or a subject at risk of or at increased risk of developing an EV-associated disease as described elsewhere herein.

The compositions and methods described herein can be administered to a subject having or diagnosed as having an EV-associated disease and/or calcification/microcalcification. In some embodiments, the methods described herein comprise administering an effective amount of compositions described herein, e.g. an agent that reduces the level or activity of ANXA1 to a subject in order to alleviate a symptom of an EV-associated disease and/or calcification/microcalcification. As used herein, “alleviating a symptom” of a disease is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of an agent that reduces the level or activity of ANXA1 needed to alleviate at least one or more symptom of the disease or disorder, and relates to a sufficient amount of pharmacological composition to provide the desired effect. The term “therapeutically effective amount” therefore refers to an amount of an agent that reduces the level or activity of ANXA1 that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for EV levels or calcification, among others. The dosage can be determined by a physician and adjusted, as necessary, to suit observed effects of the treatment.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

In some embodiments, the technology described herein relates to a pharmaceutical composition comprising an agent that reduces the level or activity of ANXA1 as described herein, and optionally a pharmaceutically acceptable carrier. In some embodiments, the active ingredients of the pharmaceutical composition comprise an agent that reduces the level or activity of ANXA1 as described herein. In some embodiments, the active ingredients of the pharmaceutical composition consist essentially of an agent that reduces the level or activity of ANXA1 as described herein. In some embodiments, the active ingredients of the pharmaceutical composition consist of an agent that reduces the level or activity of ANXA1 as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. an agent that reduces the level or activity of ANXA1 as described herein.

In some embodiments, the pharmaceutical composition comprising an agent that reduces the level or activity of ANXA1 as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of an agent that reduces the level or activity of ANXA1 as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an agent as disclosed herein can also be incorporated into the parenteral dosage forms of the disclosure, including conventional and controlled-release parenteral dosage forms.

Pharmaceutical compositions comprising an agent that reduces the level or activity of ANXA1 can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the agent that reduces the level or activity of ANXA1 can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos.: 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1 ; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

Im some embodiments of any of the aspects, the an agent that reduces the level or activity of ANXA1 described herein is administered as a monotherapy, e.g., another treatment for the EV-associated disease or calcification/microcalcification is not administered to the subject.

In some embodiments of any of the aspects, the methods described herein can further comprise administering a second agent and/or treatment to the subject, e.g. as part of a combinatorial therapy. Non-limiting examples of a second agent and/or treatment for subjects with cancer can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE®. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

Therapeutic agents for other conditions described herein are well-known in the art and can readily be identified by one of ordinary skill by consulting, e.g., a current edition of the FDA's Orange Book (Approved Drug Products with Therapeutic Equivalence Evaluations).

In certain embodiments, an effective dose of a composition comprising an agent that reduces the level or activity of ANXA1 as described herein can be administered to a patient once. In certain embodiments, an effective dose of a composition comprising an agent that reduces the level or activity of ANXA1 can be administered to a patient repeatedly. For systemic administration, subjects can be administered a therapeutic amount of a composition comprising an agent that reduces the level or activity of ANXA1, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. calcification by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active agent. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising an agent that reduces the level or activity of ANXA1 can be administered over a period of time, such as over a 5 minute, 10 minute, 15 minute, 20 minute, or 25 minute period.

The dosage ranges for the administration of an agent that reduces the level or activity of ANXA1, according to the methods described herein depend upon, for example, the form of the agent, its potency, and the extent to which symptoms, markers, or indicators of a condition described herein are desired to be reduced, for example the percentage reduction desired for calcification. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of an agent that reduces the level or activity of ANXA1 in, e.g. the treatment of a condition described herein, or to induce a response as described herein (e.g. a reduction in calcification) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. calcification. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of an EV-associated disease and/or calcification/microcalcification. When using an experimental animal model, efficacy of treatment is evidenced when a statistically significant change in a marker is observed, e.g. calcification.

The efficacy of a given dosage combination can also be assessed in an animal model, e.g. in a mouse model as described in the Examples herein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of an EV-associated disease and/or calcification/microcalcification. A subject can be male or female.

A subject can be one who has been previously diagnosed with or identified as suffering from or having a condition in need of treatment (e.g. an EV-associated disease and/or calcification/microcalcification) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

Antibodies and/or antibody reagents can include an immunoglobulin molecule, a monoclonal antibody, a chimeric antibody, a CDR-grafted antibody, a humanized antibody, a fully human antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof

As used herein, the term “nanobody” or single domain antibody (sdAb) refers to an antibody comprising the small single variable domain (WH) of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/ 02214 (WO 94/04678 published 3 Mar. 1994; which is incorporated by reference herein in its entirety).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety. These features combined with the low antigenicity to humans indicate great therapeutic potential.

As used herein, the term “nucleic acid” or “nucleic acid sequence” refers to any molecule, preferably a polymeric molecule, incorporating units of ribonucleic acid, deoxyribonucleic acid or an analog thereof The nucleic acid can be either single-stranded or double-stranded. A single-stranded nucleic acid can be one nucleic acid strand of a denatured double- stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target, e.g. ANXA1. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g,. siRNA, shRNA,miRNA, and/or amiRNA, which are well known in the art.

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and -N(CH3)-CH2—CH2-[wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Modified RNAs can also contain one or more substituted sugar moieties. The iRNAs, e.g., dsRNAs, described herein can include one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2).nOCH3, O(CH2)nNH2, O(CH2)nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)20N(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof Thus, for example, intervening untranslated yet transcribed sequences can be present between a promoter sequence and the coding sequence and the promoter sequence can still be considered “operably linked” to the coding sequence.

“Marker” in the context of the present invention refers to a molecule, structure, or an expression product , e.g., nucleic acid or polypeptide which is differentially present in a sample taken from subjects having having an EV-associated disease and/or calcification/microcalcification, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the agent described herein is exogenous. In some embodiments of any of the aspects, the agent described herein is ectopic. In some embodiments of any of the aspects, the agent described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid encoding a polypeptide as described herein or an inhibitory nucleic acid as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optomized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. an EV-associated disease and/or calcification/microcalcification. The term “treating” includes reducing or alleviating at least one adverse effect or symptom of a condition, disease or disorder associated with an EV-associated disease. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating an extracellular vesicle (EV)-associated disease in subject, the method comprising: administering an agent that reduces the levels or activity of Annexin A1 (ANXA1) in a subject in need thereof.
2. The method of paragraph 1, wherein the EV-associated disease is selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.
3. A method of reducing vascular calcification in subject, the method comprising: administering an agent that reduces the levels or activity of Annexin A1 (ANXA1) in a subject in need thereof
4. The method of paragraph 3, wherein the subject has a disease selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.
5. A method for inhibiting or reducing trafficking of an extracellular vesicle (EV) from a cell, the method comprising: contacting the cell with an agent that reduces the levels or activity of ANXA1.
6. The method of paragraph 5, wherein the cell is a smooth muscle cell (SMC), a valvular interstitial cell (VIC), oligodendroglioma cell, endothelial cell, macrophage, monocyte, or cancer cell.
7. The method of any of paragraphs 1-6, wherein the agent is an inhibitor of ANXA1.
8. The method of any of paragraphs 1-7, wherein the agent is a small molecule, nucleic acid, polypeptide, antibody reagent, or genome editing system.
9. The method of paragraph 8, wherein the nucleic acid is an inhibitory nucleic acid, silencing RNA (siRNA), microRNA (miRNA), or short hairpin RNA (shRNA).
10. The method of paragraph 9, wherein the siRNA sequence comrpises SEQ ID NO: 1 or 2.
11. The method of paragraph 8, wherein the antibody reagent is an anti-ANXA1 antibody reagent.
12. The method of paragraph 11, wherein the antibody reagent is an ANXA1 neutralizing antibody reagent.
13. The method of paragraph 12, wherein the ANXA1 neutralizing antibody reagent is an N-terminal ANXA1 neutralizing antibody reagent.
14. The method of paragraph 8, wherein the small molecule is a calcium chelator.
15. The method of paragraph 14, wherein the calcium chelator is ethylenediaminetetraacetic acid (EDTA).
16. The method of any of paragraphs 1-15, wherein the agent reduces ANXA1 loading into the extracellular vesicles.
17. The method of paragraph 16, wherein the agent is an inhibitor of dynamin-related protein 1 (DRP1).
18. The method of paragraph 17, wherein the agent is mdivi-1 or is an inhibitory nucleic acid.
19. The method of any of paragraphs 1-18, wherein the agent induces cleaves of the N-terminal domain of ANXA 1.
20. The method of paragraph 19, wherein the agent is an agonist of proteinase 3 or HLE.
21. The method of any of paragraphs 1-20, wherein the subject is a mammal.
22. The method of paragraph 21, wherein the subject is a human.
23. The method of any of paragraphs 1-22, the method further comprising receiving the results of an assay that indicates that the subject has an increase in the level of microcalcifications before administering the agent that reduces the levels or activity of ANXA 1.
24. The method of paragraph 23, further comprising, receiving the results of an assay that indicates that the subject has an increase in the level or activity of ANXA1.
25. The method of paragraph 23 or 24, wherein the assay is selected from the group consisting of: an extracellular vesicle calcification assay; a vesicle binding assay; a vesicle tethering assay; a tissue non-specific alkaline phosphatase (TNAP) assay; an immunohistochemistry assay; mass spectrometry; proteomics; scanning electron microscopy; and transmission electron microscopy.
26. The method of paragraph 25, wherein the biological sample is blood, a vascular tissue, or a heart valve tissue.
27. One or more agents that reduce the levels or activity of Annexin A1 (ANXA1) for use in treating an extracellular vesicle (EV)-associated disease in subject.
28. The one or more agents of paragraph 27, wherein the EV-associated disease is selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.
29. One or more agents that reduce the levels or activity of Annexin A1 (ANXA1) for use in reducing vascular calcification in subject.
30. The one or more agents of paragraph 29, wherein the subject has a disease selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.
31. The one or more agents of any of paragraphs 27-30, wherein the agent is an inhibitor of ANXA1.
32. The one or more agents of any of paragraphs 27-31, wherein the agent is a small molecule, nucleic acid, polypeptide, antibody reagent, or genome editing system.
33. The one or more agents of paragraph 32, wherein the nucleic acid is an inhibitory nucleic acid, silencing RNA (siRNA), microRNA (miRNA), or short hairpin RNA (shRNA).
34. The one or more agents of paragraph 33, wherein the siRNA sequence comrpises SEQ ID NO: 1 or 2.
35. The one or more agents of paragraph 32, wherein the antibody reagent is an anti-ANXA1 antibody reagent.
36. The one or more agents of paragraph 35, wherein the antibody reagent is an ANXA1 neutralizing antibody reagent.
37. The one or more agents of paragraph 36, wherein the ANXA1 neutralizing antibody reagent is an N-terminal ANXA1 neutralizing antibody reagent.
38. The one or more agents of paragraph 32, wherein the small molecule is a calcium chelator.
39. The one or more agents of paragraph 38, wherein the calcium chelator is ethylene diaminetetraacetic acid (EDTA).
40. The one or more agents of any of paragraphs 27-39, wherein the agent reduces ANXA1 loading into the extracellular vesicles.
41. The one or more agents of paragraph 40, wherein the agent is an inhibitor of dynamin-related protein 1 (DRP1).
42. The one or more agents of paragraph 41, wherein the agent is mdivi-1 or is an inhibitory nucleic acid.
43. The one or more agents of any of paragraphs 27-42, wherein the agent induces cleaves of the N-terminal domain of ANXA 1.
44. The one or more agents of paragraph 43, wherein the agent is an agonist of proteinase 3 or HLE.
45. The one or more agents of any of paragraphs 27-44, wherein the subject is a mammal.
46. The one or more agents of paragraph 45, wherein the subject is a human.
47. The one or more agents of any of paragraphs 27-46, wherein the one or more agent is administered to a subject determined to have an increase in the level of microcalcifications.
48. The one or more agents of any of paragraphs 27-47, wherein the one or more agent is administered to a subject determined to have an increase in the level or activity of ANXA1.
49. The one or more agents of any of paragraphs 47-48, wherein the determination is made using an assay selected from the group consisting of: an extracellular vesicle calcification assay; a vesicle binding assay; a vesicle tethering assay; a tissue non-specific alkaline phosphatase (TNAP) assay; an immunohistochemistry assay; mass spectrometry; proteomics; scanning electron microscopy; and transmission electron microscopy.
50. The one or more agents of any of paragraphs 47-49, wherein the level is the level in a biological sample.
51. The one or more agents of paragraph 50, wherein the biological sample is blood, a vascular tissue, or a heart valve tissue.

### EXAMPLES

### Example 1

### Annexin A1 Tethering Aggregates Human Extracellular Vesicles and Promotes Cardiovascular Calcification

Cardiovascular cells secrete extracellular vesicles (EVs) that aggregate in extracellular matrix and form microcalcifications associated with atherosclerotic plaque rupture and heart valve failure. How EVs aggregate in cardiovascular and other human diseases is unknown. It was hypothesized by the authors that EVs contain tethering proteins, which promote aggregation of vesicles in the extracellular matrix leading to cardiovascular calcification. To identify the mechanisms that generate aggregated EVs, quantitative proteomics and pathway analysis were used, through which it was determined that annexin A1 (ANXA1) tethers EVs in human artery and aortic valve tissues and cells. Addition of human ANXA1 to vesicles resulted in time- and concentration-dependent aggregation that calcium chelation or ANXA1 neutralizing antibody inhibited (n=3, P<0.0001). Supporting a role of altered calcium signaling in mediating increased annexin and related calcium-binding proteins loading on EVs, treatment with the mitochondrial fission and dynamin-related protein 1 inhibitor, mdivi-1, suppressed osteogenic differentiation-induced ANXA1 loading on EVs (n=3, P<0.05). Furthermore, ANXA1 knockdown attenuated calcification of human artery (n=3, P<0.01) and aortic valve cells (n=3, P<0.05) as well as calcification aggregates of EVs derived from human arteries (n=3, P<0.001) and valves (n=3, P<0.05) cultured in 3D-collagen hydrogels. This work reveals that ANXA1 and EV aggregation are biologically and chemically targetable in ectopic microcalcification formation. More broadly, these findings provide proof-of-concept that protein-mediated tethering extends beyond intracellular trafficking vesicles. It is demonstrated that tethering of EVs occurs in the extracellular matrix independent of cells, and EV tethering is associated with human cardiovascular disease. The finding that EV tethering promotes critical cardiovascular pathology, can extend to other EV-associated diseases, including autoimmune and neurodegenerative diseases and cancer.

### Example 2

### Annexin A1 Tethering Aggregates Human Extracellular Vesicles and Promotes Cardiovascular Calcification

Cardiovascular cells secrete extracellular vesicles (EVs) that aggregate in extracellular matrix and form microcalcifications associated with atherosclerotic plaque rupture and heart valve failure. How EVs aggregate in cardiovascular and other human diseases is unknown. It was hypothesized by the inventors that EVs contain tethering proteins, which promote aggregation of vesicles in the extracellular matrix leading to cardiovascular calcification. To identify the mechanisms that generate aggregated EVs, quantitative proteomics and pathway analysis was used through which it was determined that annexin A1 (ANXA1) tethers EVs in human artery and aortic valve tissues and cells. Addition of human ANXA1 to vesicles resulted in time- and concentration-dependent aggregation that calcium chelation or ANXA1 neutralizing antibody inhibited significantly. Supporting a role of altered calcium signaling in mediating increased annexin and related calcium-binding proteins loading on EVs, treatment with the mitochondrial fission and dynamin-related protein 1 inhibitor, mdivi-1, suppressed osteogenic differentiation-induced ANXA1 loading on EVs. Furthermore, ANXA1 knockdown attenuated calcification of human artery and aortic valve cells as well as calcification aggregates of EVs derived from human arteries and valves cultured in 3D-collagen hydrogels. This work reveals that ANXA1 and EV aggregation are biologically and chemically targetable in ectopic microcalcification formation. More broadly, these findings provide proof-of-concept that protein-mediated tethering extends beyond intracellular trafficking vesicles. It is demonstrated that tethering of EVs occurs in the extracellular matrix independent of cells, and EV tethering is associated with human cardiovascular disease. The finding that EV tethering promotes critical cardiovascular pathology, extends to other EV-associated diseases, including autoimmune and neurodegenerative diseases and cancer.

Proteins tether trafficking vesicles with cell membranes regulating vesicle transport inside cells and at the cell surface. Herein it is demonstrated that protein-mediated vesicle tethering is not limited to intracellular trafficking and occurs in the extracellular matrix independent of cells with extracellular vesicles (EVs) tethering each other. This process has a critical pathophysiological function as tethering promotes EV aggregation, which drives the formation of microcalcification. Microcalcifications in turn cause plaque rupture and aortic valve failure. Currently, no anti-calcification drug therapies are available. These findings open an area of research involving EV tethering and pave the way for the development of therapeutic strategies targeting EV aggregation in cardiovascular diseases, and other human EV-associated diseases, including autoimmune and neurodegenerative diseases and cancer.

Introduction

Considerable molecular understanding of membrane vesicle trafficking within and between cells related to cell growth and maintenance, neurotransmission, and regulated insulin secretion has been achieved (1, 2, 3, 4, 5). Yet, the pathophysiological roles of vesicles outside of cells, termed extracellular vesicles (EVs) are less defined. Intracellularly, vesicles serve as transporters with vesicle and cell membrane proteins mediating tethering at membrane contact sites (6, 7), driving vesicle and membrane fusion (8). Whether EV tethering occurs in the extracellular matrix independent of cells, and how EV tethering contributes to the development of human diseases, particularly to calcification, is unknown.

EVs are well-established mediators of mineralization in bone, cartilage, and dentin, acting via regulation of calcium and phosphate metabolism (9). Smooth muscle cell (SMC) and valvular interstitial cell (VIC)-derived EVs serve as vascular (10, 11) and valvular (12) calcification nucleation sites, as such the mechanisms by which EVs generate microcalcifications hold great clinical interests as they contribute to arterial plaque rupture (13) and heart valve failure (14). Cardiovascular cells secrete EVs that aggregate and promote mineralization (10), but how EVs aggregate and generate ectopic calcification is unclear. Established EV cargos involved in matrix mineralization, include the phosphate metabolizing enzyme tissue non-specific alkaline phosphatase (TNAP), and annexins A2, A5, and A6 that act as calcium transporters involved in the formation of hydroxyapatite mineral (11, 15, 16, 17). Annexin A5 (ANXAS) is involved in the binding of EVs to collagen type II and X in cartilage (18), but human ANXAS does not aggregate lipid membranes (19). The mechanisms that induce the binding and aggregation of EVs to each other in the extracellular matrix have not been identified. Demonstrated herein is a new functional role of annexin in tethering and aggregating EVs on top of the roles of annexins in calcification.

In addition to cardiovascular diseases, EVs and ectopic calcification associate with autoimmune and neurodegenerative diseases and cancer. In cancer, intracranial calcification occurs in up to 90% of oligodendroglioma patients (20). Oligodendroglioma cells release EVs containing extracellular matrix degrading proteins that can contribute to malignant invasion (21). In neurodegenerative diseases, including Alzheimer's disease, extracellular matrix alterations also occur. Vascular calcification is associated with increased risk of dementia and Alzheimer's disease (22). Chemically inhibiting EV secretion reduces amyloid plaques associated with Alzheimer's disease, whereas injection of EVs into mice brains promotes amyloid beta aggregation and plaque formation (23). In autoimmune disease, ectopic calcification is observed in approximately 40% of systemic sclerosis patients (24). Human systemic sclerosis platelet-derived EVs induce inflammation, matrix changes and fibrosis in mice (25). The inventors hypothesized that EVs contain tethering proteins that promote extracellular aggregation, which drives calcific pathology in cardiovascular disease and perhaps other EV-associated diseases.

Results

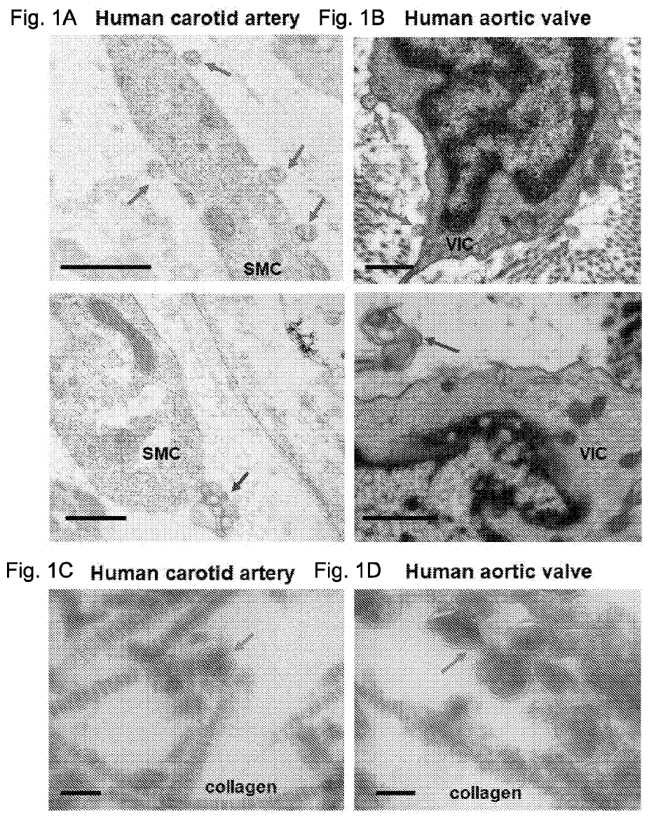

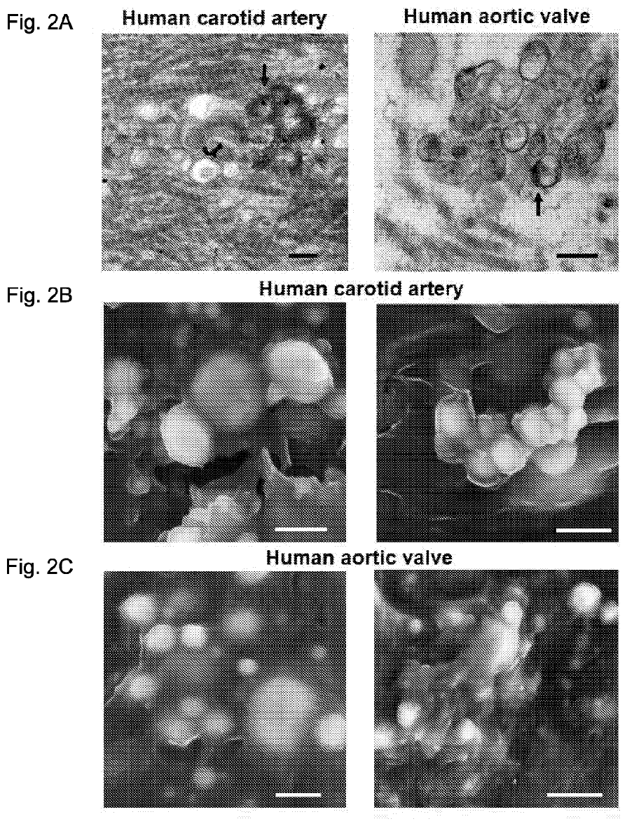

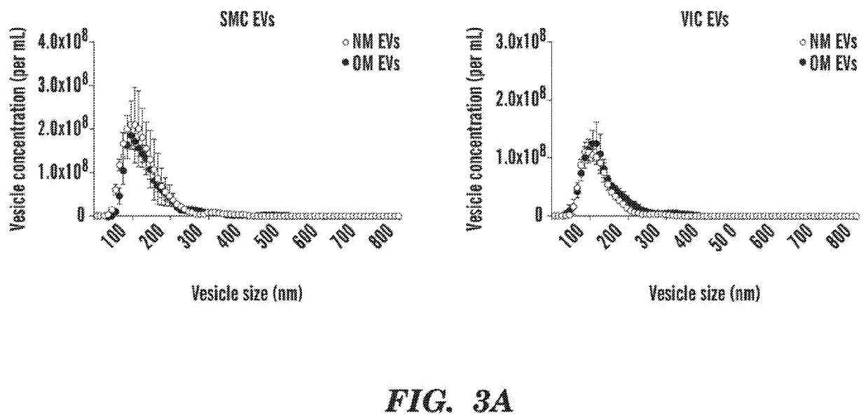

Human Cardiovascular Tissue SMCs and VICs Released EVs that Aggregated and Calcified. To determine the cellular origin of calcifying EVs, EVs in calcified human carotid artery and aortic valve tissues were examined as examples of common cardiovascular pathology. Using electron microscopy, EVs were observed originating from SMCs in calcified artery and VICs in calcified valve tissues (FIGS. 1A and 1B). In agreement with EVs being derived from the plasma membrane (26) and from exosome pathways (27) EVs were observed originating by both plasma membrane budding and multivesicular body release in human artery tissue SMCs and aortic valve tissue VICs (FIGS. 1A and 1B). Aggregated EVs were also observed independent of cells, including EVs binding to each other and not only to collagen fibers in the extracellular matrix, in both calcified human artery and valve tissues (FIGS. 1C and 1D). In agreement with an EV origin of cardiovascular microcalcification, aggregated and calcifying EVs localized within the collagen extracellular matrix of calcified human artery and valve tissues (FIG. 2A). Using density-dependent scanning electron microscopy, spherical calcification aggregates were observed in diseased human artery and valve tissues, further supporting an EV origin of cardiovascular microcalcifications (FIGS. 2B and 2C). Taken together these results demonstrate that SMCs in human artery and VICs in human aortic valve tissues release nanometer-sized EVs that aggregate in the collagen extracellular matrix by binding to each other, and then mineralize forming larger ectopic microcalcifications.

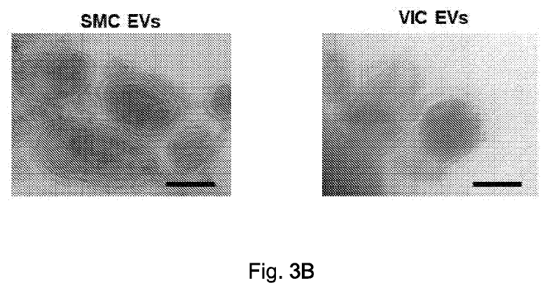

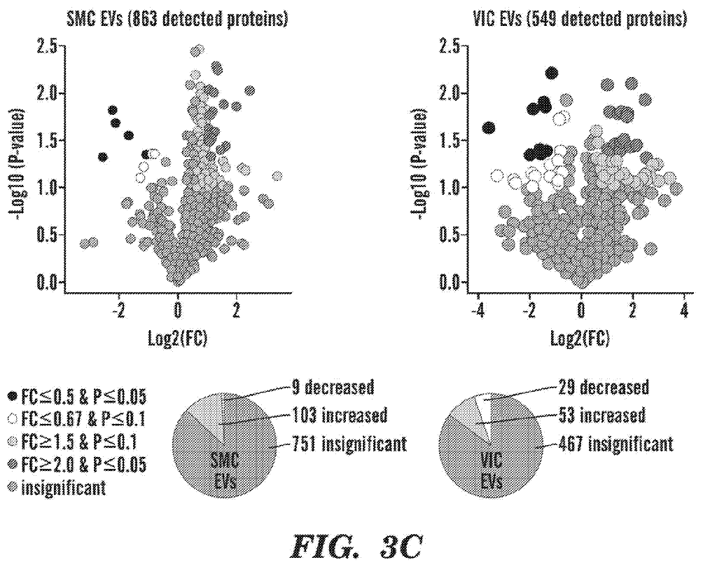

Human Cardiovascular EV Protein Composition was Altered Under Osteogenic Conditions. To investigate EV protein mechanistic contributions to calcification, it was assessed whether human vascular and valvular EV composition changed under calcifying conditions. While other cell types, including macrophages (16) can contribute to the calcification process, SMC- and VIC-derived EVs obtained from both normal control media (NM) and calcifying osteogenic media (OM) cultured cells were focused on. To do this, label-free quantitative proteomics was used to assess human vascular and valvular cell EV protein composition in calcification-promoting OM. No differences were measured by nanoparticle tracking analysis for human coronary artery SMC- and aortic VIC-derived EV abundance or size (ranging from about 50-300 nm) in OM compared to NM (FIG. 3A). Electron microscopy was used to further visually validate that the proteomics starting material was composed of EVs from both SMCs and VICs (FIG. 3B). Out of 863 detected proteins, 103 were increased and nine decreased in OM cultured human SMC-derived EVs (FIG. 3C). Out of 549 detected proteins, 53 were increased and 29 decreased in human VIC-derived EVs in OM (FIG. 3C). Proteomics further validated an enrichment of EVs in the samples through the identification of EV markers, including flotillin-1. The quantitative proteomics data demonstrated that protein composition changes occur in primary human SMC and VIC EVs under osteogenic conditions.

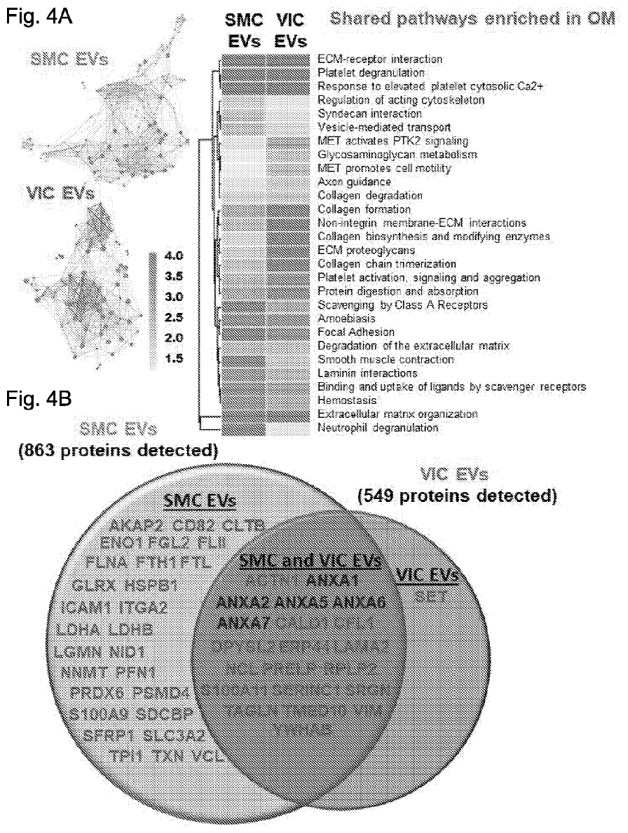

Human SMC- and VIC-Derived EVs Contained Tethering Proteins. Next, it ws examined whether EVs obtained from human SMCs and VICs had tethering proteins that could generate EV and calcification aggregates such as those observed in human cardiovascular tissues. Quantitative pathway analysis (28) was performed to triage candidate tethering proteins from the human EV proteomics datasets. Human SMC- and VIC-derived EVs in OM showed enrichment in several pathways compared to EVs in conditioned NM (data not shown). Comparative analysis revealed human SMC and VIC EVs had shared enriched pathways in OM (FIG. 4A). Annexins are associated with several of these shared enriched pathways. Annexin A1 (ANXA1) was identified in the enriched “smooth muscle contraction” pathway in the EV datasets (FIG. 4A). “Vesicle-mediated transport” pathway was enriched in SMC and VIC EVs in OM (FIG. 4A). Linking annexins to this pathway, ANXA1 mediates intracellular endosomal transport (29). Response to elevated cytosolic calcium pathway was enriched in both SMC- and VIC-derived EVs in OM (FIG. 4A). Mitochondrial dynamics and intracellular calcium signaling are altered in cardiovascular calcification (30). Annexins associate with plasma membrane in response to cytosolic calcium (31), from which they can be loaded onto EVs, suggesting a role of altered calcium signaling in annexin EV regulation in OM. Taken together, the proteomics and pathway analysis revealed human SMC- and VIC-derived EVs contain annexin proteins, including ANXA1 that could promote EV tethering.

To assess the role of annexins in mediating EV aggregation, the abundances of annexins and related binding proteins in human SMC- and VIC-derived EVs under calcifying conditions were compared. Annexins A3, A4, and A 11 were detected in both SMC and VIC EVs but were not altered in OM. ANXA1 was increased in SMC EVs and was detected in VIC EVs in OM (FIG. 4B). In addition to annexins, comparative proteomics analysis revealed 16 upregulated proteins out of 529 detected in both SMC and VIC EVs in OM, including an ANXA1 calcium-dependent binding partner, S100 calcium binding protein A11 (S100A11) (FIG. 4B). Supporting a role of S100A11 in promoting annexin activity, S100A11-ANXA1 complexing is required for proper ANXA1-mediated endosomal trafficking in HeLa cells (29). Annexin A2 (ANXA2), annexin A5 (ANXA5), and annexin A7 (ANXA7) were increased in human SMC EVs, and ANXA5 and annexin A6 (ANXA6) were increased in human VIC EVs in OM (FIG. 4B). S100A9 binds ANXA6 (32). S100A9 was increased in SMC EVs in OM (FIG. 4B). A S100A9/ANXA5/phosphatidylserine complex regulates macrophage EV calcification (16). However, whether annexins or other proteins mediate EV tethering and aggregation is unknown. ANXA5 binds to collagen (18), but human ANXA5 does not tether lipid membranes (19); although, several other annexins aggregate in vitro synthesized membrane (19). ANXA2 and ANXA6 associate with vascular (11, 15) and valvular (33) vesicle-mediated calcification, in part by acting as calcium channels, which can also regulate tissue-nonspecific alkaline phosphatase (TNAP) activity (15). TNAP is a key mediator of osteogenic differentiation and is loaded into cardiovascular cell EVs in OM (17). The comparative proteomics data indicates that ANXA1, ANXA2, ANXA6, ANXA7, and associated S100 calcium binding proteins could promote EV tethering and TNAP activity in OM. The study showed that annexin-mediated tethering and vesicle aggregation could occur both with and without increased ANXA1 content, as increased S100 calcium and annexin binding proteins in EVs could also induce annexin tethering activity. As ANXA1 was highlighted by our pathway analysis and has not been previously demonstrated to regulate vascular or valvular calcification, the mechanistic roles of ANXA1 were assessed in EV tethering, aggregation, and microcalcification formation.

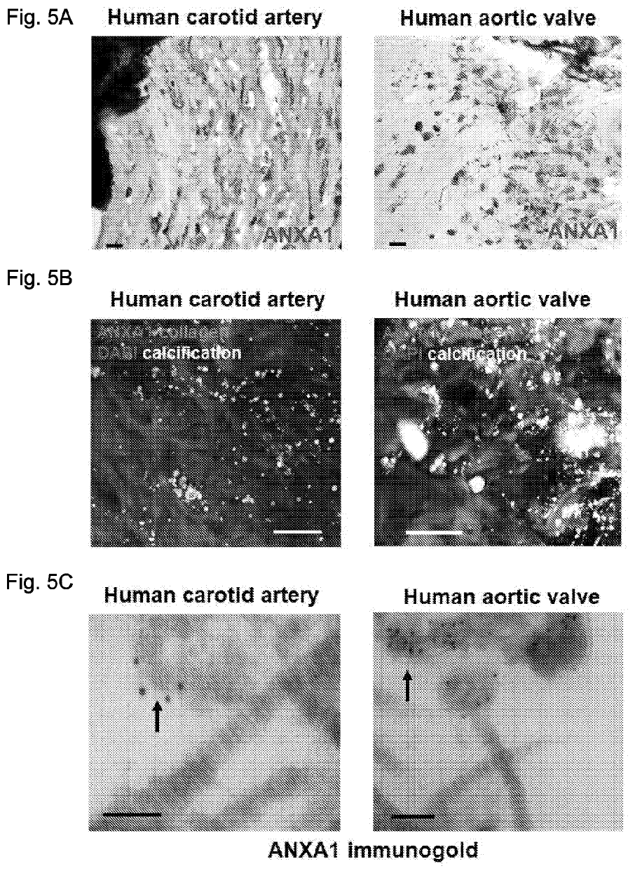

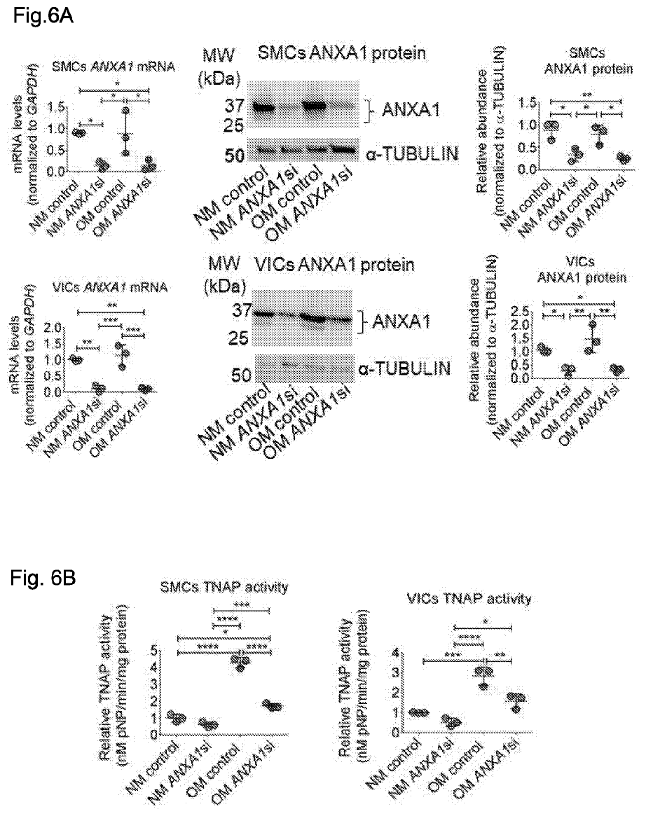

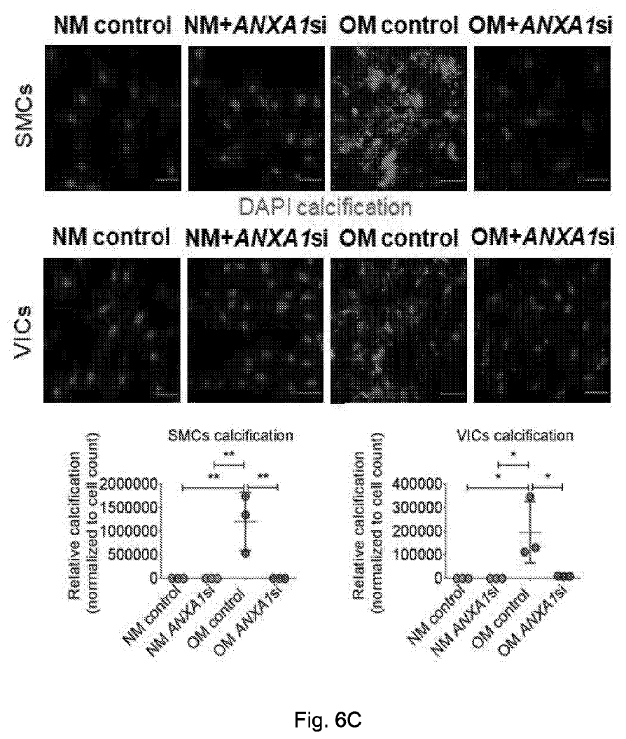

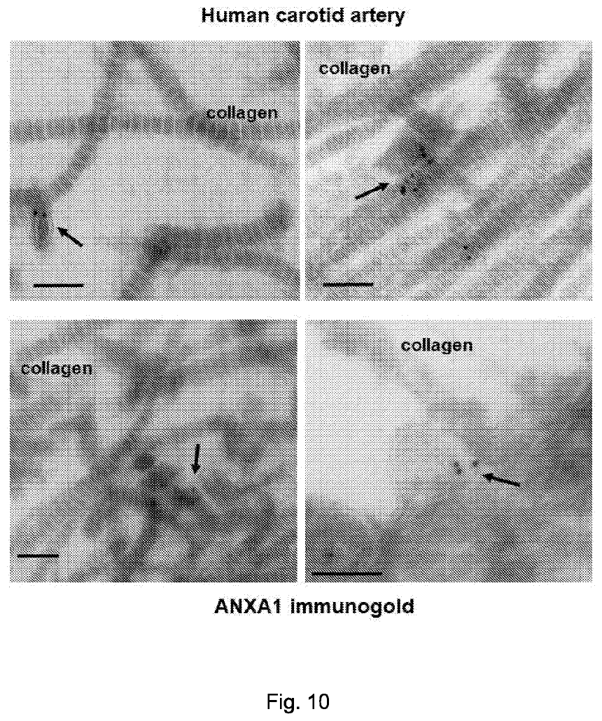

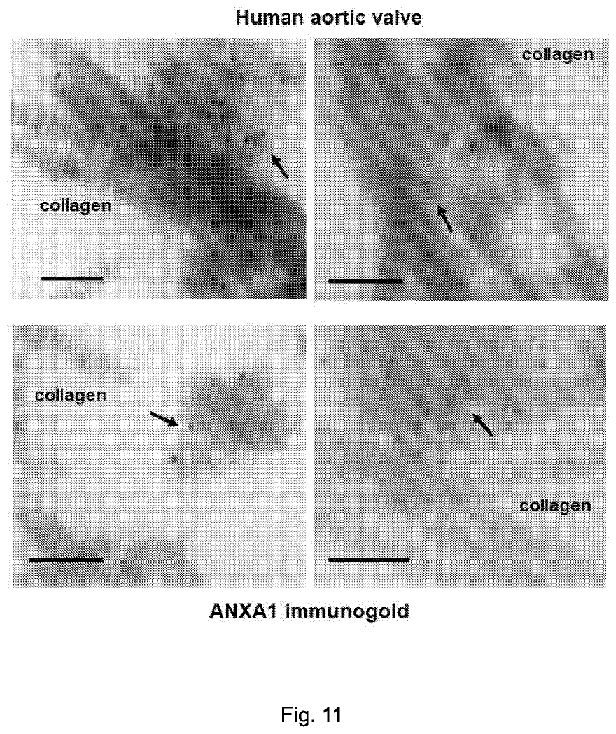

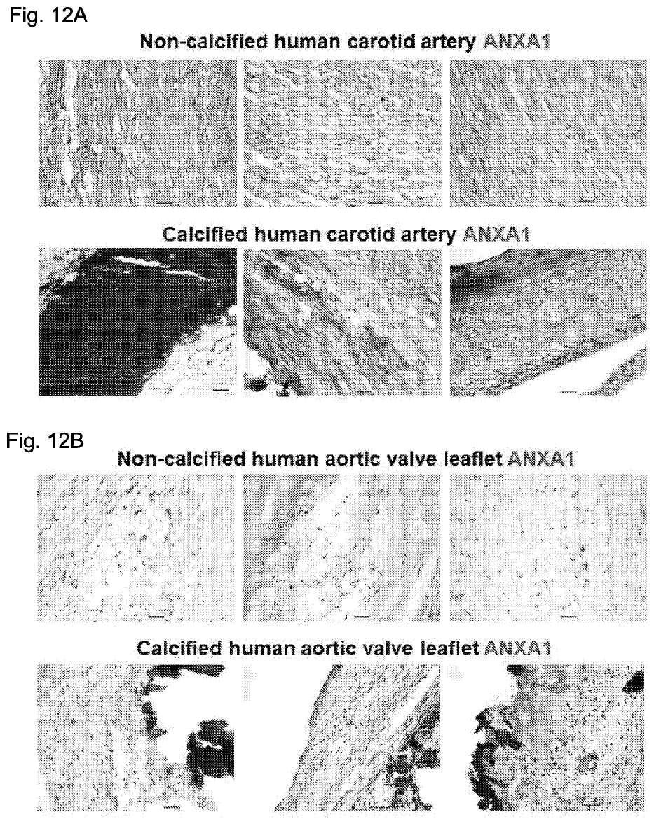

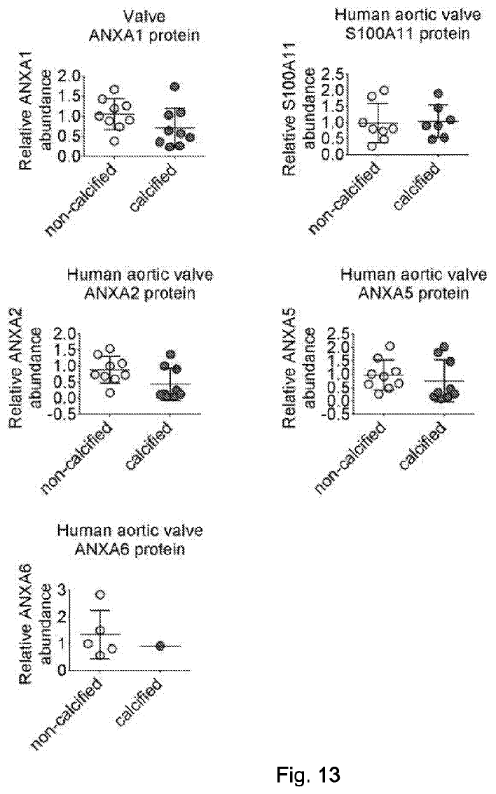

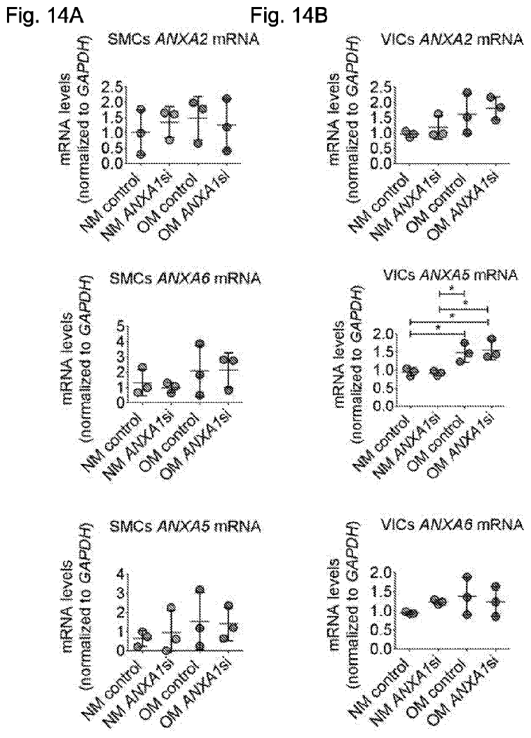

ANXA1 Localized to EVs in Calcified Human Cardiovascular Tissues. To localize ANXA1 ex vivo, immunostaining was performed on non-calcified and calcified human carotid artery and aortic valve tissues. Calcified human carotid artery and aortic valve tissues contained immunoreactive ANXA1 (FIG. 5A). ANXA1 presence near aggregated microcalcifications in human carotid artery and aortic valve extracellular matrix was confirmed using ANXA1 immunofluorescence with near-infrared calcium tracer (OsteoSense680) (34) and the collagen probe CNA35-OG488 (35) (FIG. 5B). To evaluate ANXA1-mediated EV tethering in calcified human cardiovascular tissue immunogold electron microscopy was used. Both calcified human carotid artery and aortic valve tissues showed enrichment in ANXA1 on tethered EVs (FIG. 5C, FIG. 10, FIG. 11). ANXA1 was also observed in non-calcified human artery and valve tissues (FIGS. 12A and 12B). It was validated that the total abundance of annexins is not changed between calcified and non-calcified human cardiovascular tissues, using previously reported untargeted proteomics dataset (28), which showed no statistical differences in the total abundance of S100A11, ANXA1, ANXA2, ANXAS, or ANXA6 in calcified human valve tissue compared to non-calcified valve tissue (FIG. 13). In agreement, ANXA1, ANXA2, and ANXA6 mRNA levels were not significantly altered in SMCs or VICs cultured in OM compared to NM (FIG. 6A, FIG. 14A, 14B). These data indicated that trafficking changes of calcium binding proteins most likely account for increased annexin and related binding partners' protein abundance on EVs under osteogenic conditions rather than transcriptional changes.

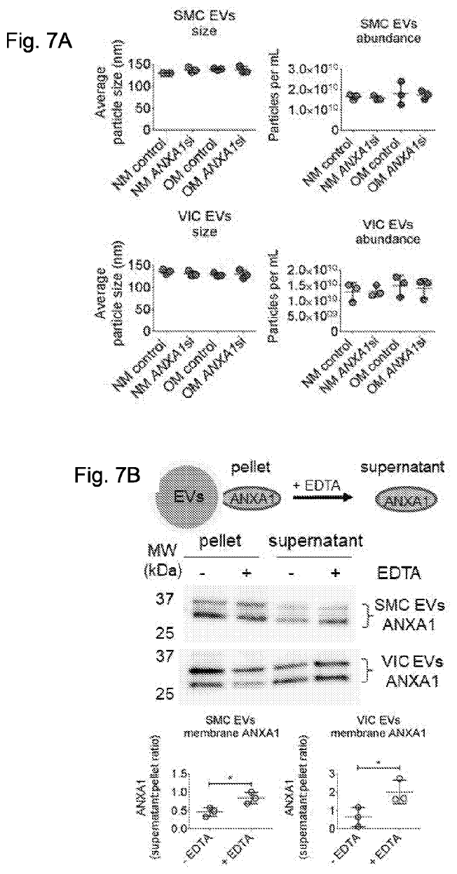

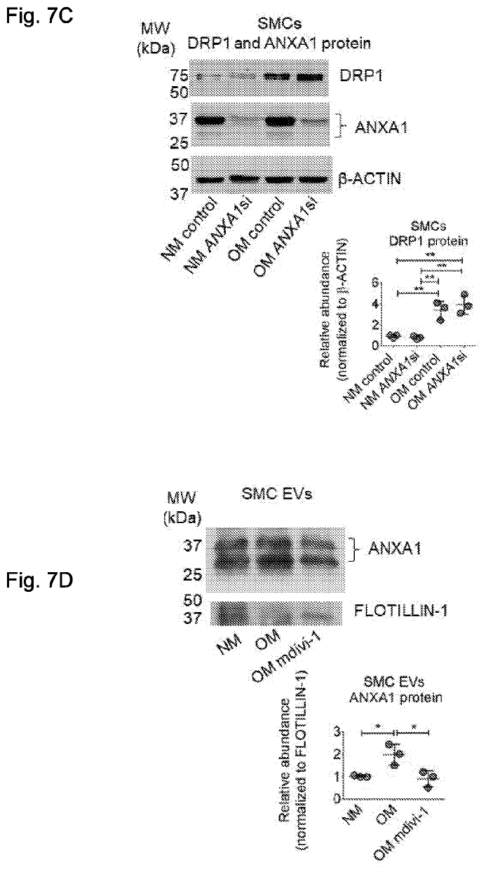

ANXA1 Knockdown Attenuated Human SMC and VIC Calcification. To assess the role of ANXA1 in cardiovascular cell calcification, ANXA1 was knocked down in human cardiovascular cells undergoing osteogenic differentiation. Primary human SMCs and VICs had significantly reduced ANXA1 mRNA levels and ANXA1 protein (FIG. 6A), without altering other annexin mRNA levels (FIG. 14A, 14B). Supporting annexin calcium channel regulation of TNAP activity (15), ANXA1 siRNA suppressed OM-induced TNAP activation in human SMCs and VICs (FIG. 6B). In addition, ANXA1 siRNA attenuated microcalcification formation in OM, visualized with a near-infrared calcium tracer in cultured human SMCs and VICs (FIG. 6C), without altering EV size or abundance (FIG. 7A). Taken together this data demonstrates that ANXA1 is a regulator of human SMC and VIC calcification in OM.

ANXA1 is a Calcium-Dependent Human Cardiovascular EV Tethering Protein. To demonstrate ANXA1 as an EV tethering protein several in vitro biochemical approaches were employed. It was first validated that ANXA1 localized on the surface of both SMC- and VIC-derived EVs, and could therefore tether EVs, by incubating EVs with ethylenediaminetetraacetic acid (EDTA) to chelate calcium and release surface bound ANXA1. Addition of EDTA to human SMC- and VIC-derived EVs led to increased ANXA1 release from EVs (FIG. 7B).

Next, as the data supported trafficking changes rather than transcriptional changes in mediating increased annexin and related calcium binding proteins on EVs, we assessed calcium regulation of ANXA1 EV loading. Inhibition of dynamin-related protein 1 (DRP1) with the small molecule inhibitor, mdivi-1 or DRP1 siRNA suppressed SMC and VIC calcification, reduced mitochondrial fragmentation, and increased intracellular calcium storage in OM (30). Therefore, it was tested whether modulation of mitochondrial calcium storage could impact annexin loading onto EVs, as annexin trafficking to the plasma membrane is mediated by cytosolic calcium binding (31). Human SMCs cultured in OM were used as a proof-of-concept model and validated that OM increased DRP1, a mitochondrial fission protein compared to NM conditions (FIG. 7C). ANXA1 siRNA did not alter DRP1 protein abundance (FIG. 7C), supporting the hypothesis that ANXA1 acts downstream of DRP1 regulation of calcium signaling. Addition of the mitochondrial fission inhibitor, mdivi-1, suppressed the increased ANXA1 loading on EVs in OM (FIG. 7D). Together these data demonstrate that alterations in calcium storage, such as those occurring during cardiovascular osteogenic differentiation, lead to increased calcium binding proteins on EVs.

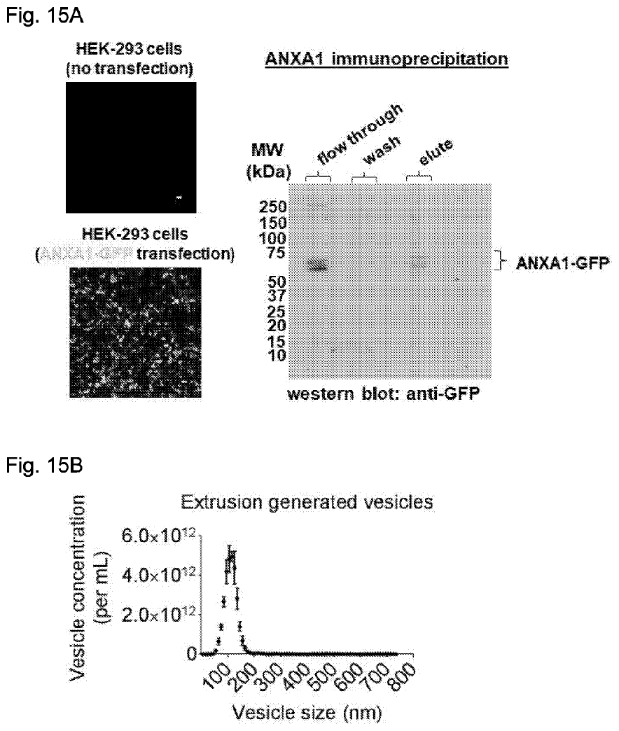

Next, ANXA1 vesicle tethering activity was validated by using two different methods. Annexins bind phosphatidylserine, a phospholipid enriched in the membranes of calcifying EVs (11, 16). Vesicles were first generated that could be visualized using confocal microscopy by swelling phosphatidylserine on polyvinyl alcohol coated cover slips. Green fluorescence protein (GFP)-tagged human ANXA1 was immunopurified to incubate with these swelling generated vesicles (FIG. 15B). Incubating GFP-tagged ANXA1 with phosphatidylserine vesicles revealed enriched ANXA1 at tethered vesicle membrane contact sites (FIG. 15A).

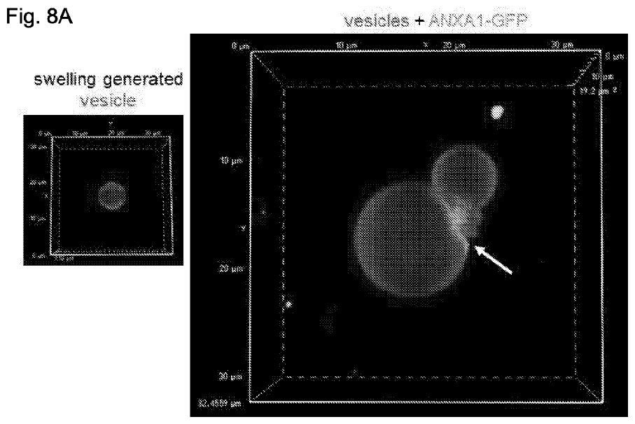

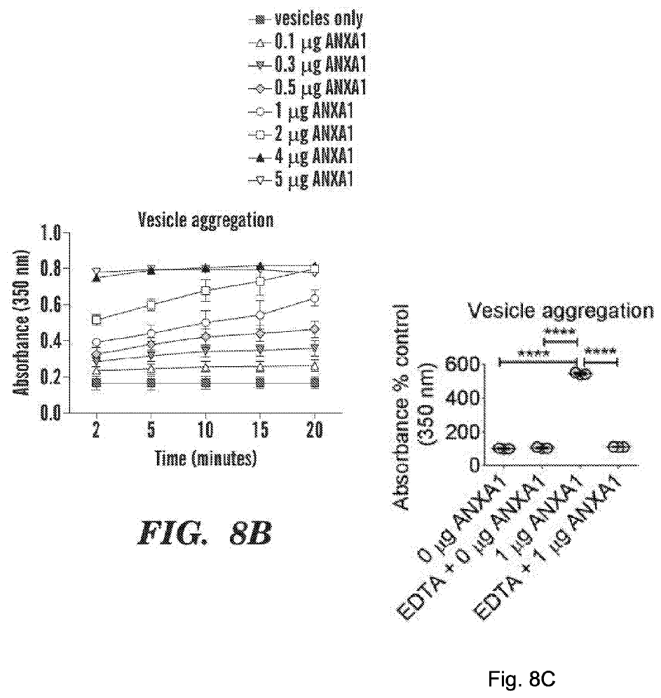

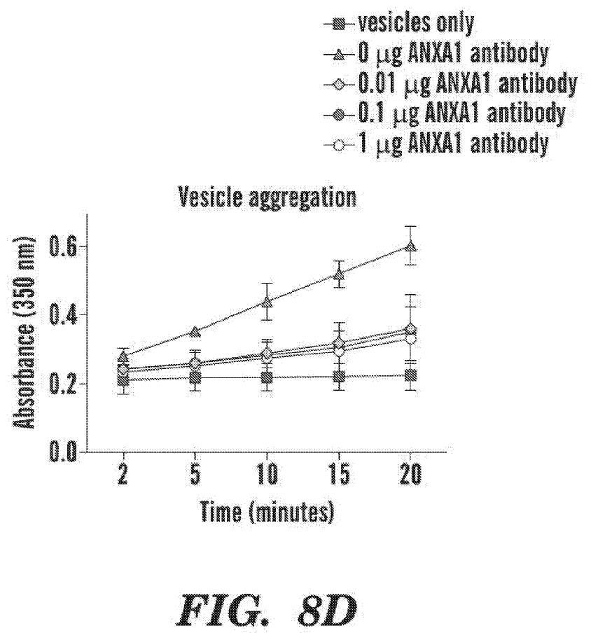

As human cardiovascular cell EVs are generally around 150 nm in diameter (10), it was next confirmed that recombinant human ANXA1 could tether and aggregate phosphatidylserine vesicles around 150 nm in diameter. In a second tethering assay, phosphatidylserine vesicles were generated by filter extrusion and verified to be around 150 nm in diameter by nanoparticle tracking analysis (FIG. 15B). Addition of human ANXA1 protein to extrusion generated vesicles led to both time- and concentration-dependent vesicle aggregation, assessed by measuring turbidity with a 96-well plate reader (FIG. 15B). As annexins bind calcium, calcium regulation of annexin membrane interaction and calcium-altered EV charge could impact annexin-mediated EV tethering. Physiologic levels of calcium at 1 mmol/L (36) in the vesicle buffer without the addition of ANXA1 did not induce vesicle aggregation (FIG. 15B). This result demonstrates that factors in addition to physiologic levels of calcium, such as annexins, are required for vesicle tethering and aggregation. Demonstrating calcium dependence for ANXA1-mediated vesicle tethering, addition of the calcium chelator, ethylenediaminetetraacetic acid (EDTA) attenuated ANXA1-mediated vesicle aggregation (FIG. 8C). Demonstrating ANXA1 specificity, vesicle aggregation was also suppressed with addition of a N-terminal ANXA1 neutralizing antibody (FIG. 8D). Together these data demonstrate that ANXA1 is a calcium-dependent human cardiovascular EV tethering protein.

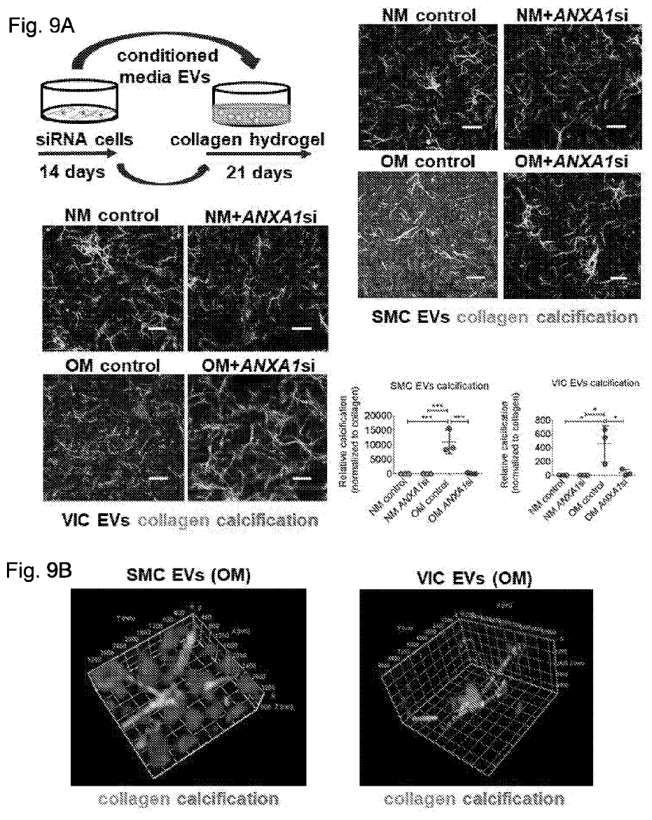

ANXA1 Knockdown Attenuated Human Cardiovascular EV-Generated Microcalcification in 3D-Collagen Hydrogels. Lastly, it was tested whether ANXA1 knockdown could suppress human EV-mediated microcalcification formation. As ANXA1 siRNA did not alter abundance or size of human SMC and VIC EVs released into conditioned media (FIG. 7A), collagen was likely involved in trapping secreted EVs that go on to tether, aggregate, and form microcalcifications. As such, to assess the aggregation and calcification potential of EVs following ANXA1 knockdown, EVs were incubated in cell-free 3D-collagen hydrogels, which were previously used to study formation and growth of EV-derived microcalcifications (10). EVs in conditioned media derived from human SMCs and VICs cultured in OM generated microcalcifications in 3D-collagen hydrogels that were attenuated by ANXA1 siRNA (FIG. 9A). It was further demonstrated EV tethering produced aggregated microcalcification formation by using super resolution microscopy. Calcified vesicle aggregates were observed with OM-conditioned media EVs derived from both human SMCs and VICs (FIG. 9B). Together these data demonstrate that ANXA1 promotes tethering, aggregation, and formation of microcalcifications from human cardiovascular EVs.

Discussion

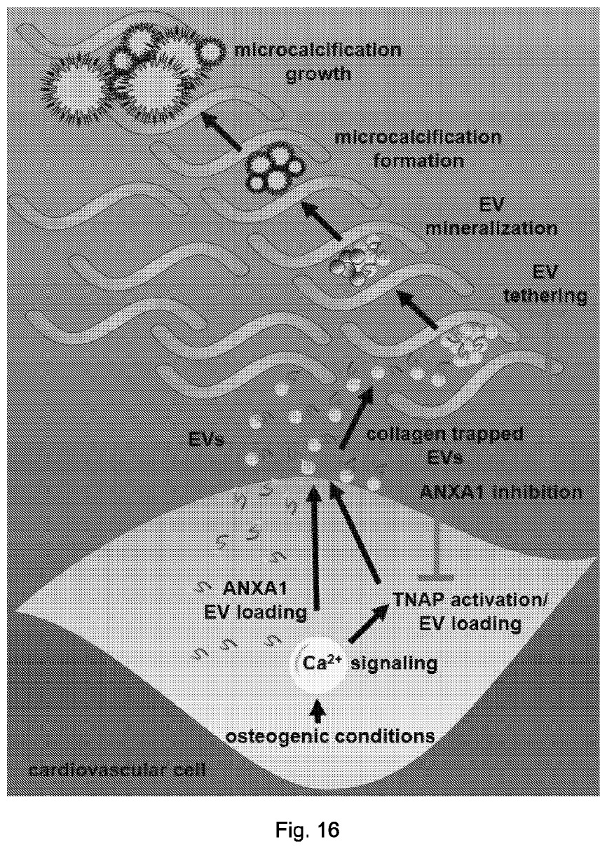

Described herein are the following new findings: 1) vesicle tethering is not limited to cellular trafficking, but also occurs in the extracellular matrix independent of cells; 2) human cardiovascular tissues generate EVs containing tethering proteins, including ANXA1 that promote EV aggregation in the extracellular matrix; 3) inhibition of mitochondrial fission suppressed ANXA1 EV loading in OM; 4) chemical and biological inhibition of ANXA1 tethering suppressed vesicle aggregation; 5) ANXA1 inhibition suppressed SMC and VIC calcification in 2D-cell culture and SMC- and VIC-derived EV calcification in 3D-collagen hydrogels. Provided herein is a working model for the present finding, which can be applied to other tissue-derived EVs (FIG. 16): Osteogenic conditions altered calcium storage and signaling (30), which induced TNAP activation via annexin calcium channels (15) and promoted TNAP loading onto EVs (17). Calcium signaling alterations increased annexin trafficking to cell membranes, leading to the incorporation of annexins and annexin-interacting proteins on EVs. EVs get trapped in the collagen extracellular matrix (10). Increased calcium binding and annexin-interacting proteins on EVs further induce EV annexin tethering. EVs trapped in collagen extracellular matrix are tethered by ANXA1, promoting the aggregation of EVs leading to EV mineralization and the formation and growth of microcalcifications.

Understanding how aggregates form is a fundamental area of research extending from physical science to biological science. In biological science, aggregation of nanometer-sized EVs explains the origin of micrometer-sized spherical ectopic calcifications that we observed in diseased human artery and valve tissues. By identifying tethering proteins on calcifying EVs we provide a mechanistic explanation of how calcification-promoting EV aggregation occurs. In addition to protein-driven tethering, physical changes including EV charge state may play a role in aggregation. EVs are generally negatively charged, and high negatively charged EVs would suppress aggregation (37). Calcifying cardiovascular EVs show enrichment in phosphatidylserine, that bears a negative charge (11, 16). Demonstrating a role of ANXA1 in physiologic EV tethering, phosphatidylserine vesicle aggregation was not observed without the addition of ANXA1 in 1 mmol/L calcium, the physiologic concentration of free ionized calcium in human blood (36). Annexins and associated S100 calcium binding proteins could further act to lower EV negative charge by binding and taking up supraphysiological amounts of positively charged calcium ions to increase EV aggregation potential. DRP1 inhibition attenuated SMC and VIC calcification, suppressed mitochondrial fragmentation and increased intracellular calcium storage in OM (30). The present study provides additional mechanistic explanation for those results by demonstrating DRP1 and cellular calcium signaling regulated ANXA1 trafficking in OM. ANXA1, ANXA2, ANXA5, ANXA6, and ANXA7 are ubiquitously expressed (38). While annexins have been shown to be increased in EVs derived from calcifying cardiovascular cells (11, 15, 16, 33), there is a lack of mechanistic explanation defining the factors that lead to this induction in EV annexin abundance. It is demonstrated herein that changes in trafficking explain how annexins and related calcium binding proteins are increased on EVs and drive disease pathology in the absence of annexin transcriptional upregulation in calcifying cardiovascular cells.

Of related interest, matrix-degrading enzymes were detected in EVs and amyloid beta was detected in human SMC-derived EVs. Therefore, tethered EVs could be involved in Alzheimer's-related amyloid seeding as an early pathology step accelerating the aggregation of amyloid beta and formation into amyloid beta plaques.

It is demonstrated herein that the functional roles of annexins, namely ANXA1, are more complex than formerly thought. The present study adds a previously undescribed functional role of ANXA1 involving EV tethering and aggregation to the well-established roles of annexins in calcification. Prior to our present study, a role of annexins or any other proteins in promoting EV tethering and aggregation independent of cells, and a role of ANXA1 in mediating cardiovascular calcification had not been demonstrated. Understanding the mechanisms by which this aggregation process occurs could lead to the development of early anti-calcification therapies. Whether additional proteins aggregate EVs independent of cells is unknown. Tetherin (also called BST2) protein can tether multivesicular bodies to plasma membrane in HeLa cells treated with the vacuolar acidification and autophagy inhibitor, bafilomycin (47). Autophagy inhibition can increase EV release in high-phosphate induced SMC calcification (48). Increased EV release in response to increased calcium stress promotes calcification (27). In the present experimental conditions, SMC and VIC EVs were not increased in OM, supporting a change in the EV cargo rather than EV quantity in driving osteogenic differentiation-mediated calcification. A change in tetherin protein abundance was not detected in SMC- and VIC-derived EVs in OM.

Translation of tethering inhibition is not trivial, as annexins have beneficial functions, including ANXA1 inflammation resolution demonstrated in atherosclerotic mice (49), and induction of microglial amyloid beta phagocytosis (46). It was found that treatment with an N-terminal ANXA1 antibody was able to suppress ANXA1-mediated vesicle tethering. The N-terminus of ANXA1 also mediates ANXA1 anti-atherosclerosis effects in hypercholesterolemic mice (49). Targeting ANXA1 cleavage to inhibit EV aggregation while simultaneously promoting the inflammation resolution properties may provide added benefits in cardiovascular disease. Increasing the cleavage and release of the ANXA1 N-terminal signaling peptide is one such way to accomplish this.

Methods

Human Tissue. Human atherosclerotic carotid artery specimens derived from endarterectomy (Brigham and Women's Hospital Institutional Review Board protocol #1999P001348), and autopsy artery samples were obtained from Brigham and Women's Hospital (Institutional Review Board protocol #2013P002517/BWH). Human aortic valve tissue was obtained from patients undergoing valve replacement (Institutional Review Board protocol #2011P001703). Written informed consent was obtained for the human tissues used in this study. Samples were transferred promptly from the operating room on ice and further processed within 30 minutes of surgical extraction. Samples were visually assessed as calcified or non-calcified tissue by hematoxylin and eosin staining. Calcified samples were further validated by incubating sections with OsteoSense680 (Perkin Elmer, Waltham, Mass.; 1:100), near-infrared based bisphosphonate calcium tracer, for 1 hour at room temperature.

Transmission Electron Microscopy. Transmission electron microscopy was performed at the Massachusetts General Hospital Program in Membrane Biology Electron Microscopy Core using a JEOL™ 1011 electron microscope. For morphology analysis, freshly isolated human tissue was collected directly after surgical removal and was fixed with 2% glutaraldehyde in 0.1M sodium cacodylate buffer. Tissue were fixed for 24 hours at room temperature. Fixative was decanted, and tissues were rinsed three times with 0.1 M sodium cacodylate buffer. For immunogold electron microscopy, tissues were fixed in 4.0% paraformaldehyde with 0.2% glutaraldehyde in 0.1 M sodium cacodylate buffer for 24 hours at room temperature, and then incubated with annexin A1 (ANXA1) antibody (Abcam, Cambridge, Mass., # ab214486). For EV analysis, conditioned cell culture media was collected and centrifuged at 1,500 rpm for 5 minutes to remove cell debris. Supernatant was collected and EVs were pelleted by ultra-centrifugation (Optima MAX-XP™, Beckman Coulter, Brae, Calif.) at 100,000 g for 40 minutes at 4° C. (TLA 120.2 rotor, Beckman Coulter). EVs pellets were fixed with 2% glutaraldehyde in 0.1M sodium cacodylate buffer for two hours, washed with 0.1 M sodium cacodylate buffer, and processed similarly to human tissues.

Density-Dependent Scanning Electron Microscopy. Density-dependent scanning electron microscopy was performed at University College London as previously described (10). Briefly, human carotid artery or aortic valve sections on glass slides were secured to aluminum sample holders with carbon tape, and silver paint was applied to the area immediately surrounding each sample. Samples were then coated with 5 nm carbon (Quorum Technologies Turbo-Pumped Thermal Evaporators model K975X, Lewes, United Kingdom). Following coating, samples were imaged on a scanning electron microscope (SEM Zeiss VP), operated at 10 kV, and equipped with both an inlens detector that recorded secondary electrons and a backscatter electron detector. Images were obtained by imaging a region in inlens mode and subsequently imaging the same region in backscatter mode. Adobe Photoshop™ CC 2018 was used on stacked images, and the inlens image was assigned to the green channel whereas the backscatter image was assigned to the red channel

Primary Cell Culture. Primary human coronary artery SMCs were obtained from Promocell (Heidelberg, Germany) and expanded in SMC Growth Medium 2 (Promocell) supplemented with epidermal growth factor (0.5 ng/mL), insulin (5 μg/mL) basic fibroblast growth factor- B (2 ng/mL), and 5% fetal bovine serum. Primary human VICs were obtained from human aortic valve tissue using collagenase digestion (28, 30). Tissue-derived SMCs were confirmed as alpha smooth muscle actin-positive by FACS (30). Tissue-derived VICs were confirmed as vimentin positive by immunohistochemistry (28). Cells were cultured at 37° C. (5% CO2, 90% humidity) and used between passages 1 and 5. Fully confluent control SMCs or VICs were cultured for a total of 14-21 days in DMEM containing 4.5 g/L glucose and L-glutamine (Lonza, Walkersville, Md.), supplemented with 10% FBS and 1% penicillin-streptomycin (control condition: termed normal media, NM). Osteogenic differentiation of SMCs and VICs was induced in fully confluent cells cultured for 14-21 days in OM, which is NM with the following additions: 10 nmol/L dexamethasone, 10 mmol/L β-glycerol phosphate, and 100 μmol/L L-ascorbic acid 2-phosphate (termed osteogenic media, OM). Media was changed every three days. For mdivi-1 assays, cells were treated in similar manner to that which we previously reported (30). Briefly, mdivi-1 (Sigma; 50 mol/L) in dimethyl soulfoxide vehicle (final vehicle concentration of 0.01%) or vehicle control (0.01%) was added to cell culture media during each media change and cells were cultured for 14 days. Prior to adding culture media to cells, mdivi-1 was solubilized in the culture media by repeated steps of hard vortexing up to 2 minutes and heating at 37° C. until the compound was fully solubilized. Tissue non-specific alkaline phosphatase (TNAP) activity was analyzed after 14 days in culture using whole cell lysates and the TNAP enzyme activity assay (BioVision, Inc., Milpitas, Calif.), according to the manufacturer's protocol. Calcification was analyzed in cells after 21 days in culture by incubating culture wells with OsteoSense680 (1:100), near-infrared based bisphosphonate calcium tracer, for 24 hours at 37° C. (5% CO2, 90% humidity). Cells were then fixed in 4% paraformaldehyde for 15 minutes at room temperature. Nuclear staining for cell count analysis was performed with DAPI (Fisher Scientific), according to manufacturer's protocol. RNA knockdown was performed by using 20 nmol/L silencer select validated ANXA1 siRNA (Fisher Scientific, #4390824 assay ID s1382), or negative control silencer select siRNA (Fisher Scientific #4390843), and transfection was performed using Lipofectamine RNAi MAX (Fisher Scientific), according to manufacturer's protocol. The siRNA was added at the beginning of experiments, and with each media change every three days until sample collection.

Mass Spectrometry. Conditioned cell culture media was collected after cells were treated for 14 days in culture and centrifuged at 1,500 rpm for 5 minutes to remove cell debris. Supernatant was collected and EVs were pelleted by ultra-centrifugation (Optima MAX-XP™ Beckman Coulter, Brae, Calif.) at 100,000 g for 40 minutes at 4° C. (TLA 120.2 rotor, Beckman Coulter). Pellets were suspended in 10 μl of LYSE buffer of the PreOmics iST™ kit for subsequent proteolysis steps, according to the manufacturer's protocol (PreOmics GmbH, Planegg/Martinsried, Germany). Peptide samples were analyzed on an Orbitrap™ Fusion Lumos mass spectrometer fronted with an EASY-Spray™ Source (heated at 45° C.) and coupled to an Easy-nLC1000™ HPLC pump (Thermo Fisher Scientific). Peptides were subjected to a dual column set-up: an Acclaim PepMap RSLC C18 trap analytical column, 75 μm×20 mm (pre-column), and an EASY-Spray™ LC column, 75 μm×250 mm (Thermo Fisher Scientific). The analytical gradient was run at 300 nl/min from 5 to 21% Solvent B (acetonitrile/0.1% formic acid) for 75 minutes, 21 to 30% Solvent B for 10 minutes, followed by 10 minutes of 95% Solvent B. Solvent A was water/0.1% formic acid. Acetonitrile and water were LC-MS-grade. The Orbitrap analyzer was set to 120 K resolution, and the top N precursor ions in 3 seconds cycle time within a scan range of 375-1500 m/z (60 seconds dynamic exclusion enabled) were subjected to collision induced dissociation (CID; collision energy, 30%; isolation window, 1.6 m/z; AGC target, 1.0 e4). The ion trap analyzer was set to a rapid scan rate for peptide sequencing (MS/MS). MS/MS data were queried against the Human UniProt database (downloaded on Aug. 1, 2014) and Bovine UniProt database simultaneously (downloaded on Aug. 1, 2014), using the SEQUEST-HT search algorithm, via the Proteome Discoverer Package (version 2.2, Thermo Scientific). VIC and SMC EV data was searched separately. Bovine peptides coming from the media bovine serum in the culture media were excluded in the final extracellular vesicle protein analysis. Trypsin was set as the digestion enzyme in the software allowing up to four miss cleavages, using a 10 ppm precursor tolerance window and a 0.6 Da fragment tolerance window. Oxidation of methionine was set as variable modifications, and carbamidomethylation of cysteine was set as a fixed modification. The peptide false discovery rate was calculated using Percolator™ provided by Proteome Discoverer and peptides were filtered based on a 1.0% false discovery rate. Peptides assigned to a given protein group (master protein), and not present in any other protein group, were considered as unique and used for quantification. A minimum of two unique peptides were included for each dataset. In order to quantify peptide precursors detected in the MS1, but not sequenced from sample to sample, we enabled the ‘Feature Mapper’ node. Chromatographic alignment was done with a maximum retention time shift of 10 minutes and a mass tolerance of 10 ppm. Feature linking and mapping settings were: retention time tolerance minimum of 0 minutes, mass tolerance of 10 ppm and signal-to-noise minimum of 5. Precursor peptide abundances were based on their chromatographic intensities and total peptide amount was used for normalization. Assessment of calcified and non-calcified human valve tissue proteomics for annexins and S100A11 were performed using our previous reported non-targeted proteomics dataset (28).

Network Analysis. Pathway enrichment analysis was performed by testing the statistically significant proteins from proteomics datasets for over-representation in biological pathways by a hypergeometric test. The resulting P values were adjusted for multiple comparisons using the Benjamini and Hochberg method for controlling false discovery rate to obtain q-values. In the network representation of significantly enriched pathways (q-value<0.05), nodes represent pathways and connections (edges) between nodes represent the shared proteins between the pathways. Node size was made proportional to the significance of enrichment measured in units of -log(q-value). Edge thickness was made proportional to the number of overlapping proteins between the two connected pathways in units of the Jaccard index, which is defined as

I=s_{A}∩s_{B}/s_{A}∩s_{B}

where s_{A} and s_{B} are the set of proteins detected in proteomics that belong to pathway A and pathway B, respectively. Edges with a Jaccard index<0.1 were discarded in the visualization for clarity. For the pathway enrichment analysis, the canonical pathways from KEGG, Biocarta, and Reactome were considered.

Immunohistochemistry and Immunofluorescence. Tissues were cut into slices with 7 μm thickness, and cryosections were fixed in acetone. Two sections were analyzed per donor. Following blocking in 4% serum, sections were incubated with ANXA1 antibody (Abcam #ab214486; 1:4000). For immunohistochemistry: sections were then incubated with biotin-labeled secondary antibody (Vector Laboratories, Burlingame, Calif.), followed by incubation with streptavidin-peroxidase (KPL, Gaithersburg, Md.), and incubated in AEC solution (Dako, Lexington, Mass.). For immunofluorescence: sections were incubated with OsteoSense680™ (1:100), near-infrared based bisphosphonate calcium tracer, for 1 hour at room temperature, and CNA35-0G488 (1:50), a collagen probe, for 1 hour at room temperature prior to fixation. ANXA1 antibody treated slides were incubated with Alexa Fluor 594-labelled secondary antibody (Life Technologies, Carlsbad, Calif.; 1:200). Immunofluorescence slides were examined using a confocal microscope A1 (Nikon Instruments Inc., Melville, N.Y.), and all images were processed with Elements 3.20 software (Nikon Instruments Inc.).

Annexin Vesicle Binding. Giant unilamellar vesicles were generated using a previously described protocol (50) with some modifications. Briefly, for the confocal-based ANXA1 vesicle binding assay, 12 mm diameter cover glass circles were coated with 5% polyvinyl alcohol (Millipore, Burlington, Mass.) in water and left to dry overnight. Dried slides were then coated with 20 μl of 10 mg/mL phosphatidylserine (Avanti Polar Lipids, Alabaster, Ala.) in chloroform containing 0 1% DiR′; DiIC18(7) 1,1′-Dioctadecyl-3,3,3′,3′-Tetramethylindotricarbocyanine Iodide) (Fisher Scientific) and dried under vacuum. 1 mL of tris buffer (pH 6) was added to cover the coated cover glass circle in a 24-well plate, and giant unilaminar vesicles were formed by swelling at room temperature for 1 hour. Vesicles were detached from the cover glass by gently tapping the plate directly beneath the slide. ANXA1 tagged green fluorescent protein (ANXA1-GFP) was obtained by transfecting HEK-293 cells (ATCC, Manassas, Va.) with human ANXA1-GFP plasmid (OriGene, Rockville, Md., #RG201569). Transfected HEK-293 cells were lysed in Pierce IP lysis buffer (Fisher Scientific), and 3 mg of total protein lysate was incubated overnight with 10 μl ANXA1 antibody (Abcam #ab214486) and 50 μl Dynabeads protein A (Thermo Fisher) under rotation at 4° C. Dynabeads were washed three times in lysis buffer, and then ANXA1-GFP was eluted by adding 0.2 mmol/L glycine. Eluted proteins were diluted in Tris buffer (pH 8) and stored at −80° C. until use. 100 μl vesicles were incubated with 2 μg ANXA1-GFP and 1 mmol/L CaCl₂ (Sigma) for five minutes and then imaged on a Nikon confocal microscope. GFP laser settings were adjusted such that no GFP signal was detected in samples without ANXA1-GFP added.

Vesicle Tethering Assay. Vesicle tethering was assessed by using a previously reported protocol (19) with modifications to adjust to a 96-well plate reader format. Large unilaminar vesicles of ˜100 nm in diameter were generated by filter extrusion. Briefly, 2.5 mg of phosphatidylserine (Avanti Polar Lipids) was solubilized in 1 mL of buffer containing: 10 mmol/L Hepes (Sigma, St. Louis, Mo.), 100 mmol/L NaCl (Sigma), 100 μmon ethylenediaminetetraacetic acid (Boston BioProducts, Ashland, Mass.), pH 6. The lipid mixture was run through an extruder containing 0.1 μm polycarbonate membranes (Avanti Polar Lipids) ten times, during which point the lipid mixture went from cloudy to clear in appearance. Vesicles were assessed for correct size and abundance prior to use by NanoSight nanoparticle tracking analysis. 50 μg recombinant human ANXA1 (R&D Systems, Minneapolis, Minn.) was resuspended in 500 μl buffer made the same day as the assays were run: 300 mmol/L sucrose (Sigma), 40 mmol/L L-histidine monohydrochloride (Sigma), 1 mmol/L CaCl₂ (Sigma), 0.5 mmol/L MgCl₂ (Sigma), pH 6. In a 96-well 0 μg (referred to as “vesicles only”) up to 5 μg recombinant ANXA1 was added (1 μg was used for assays unless otherwise stated), and the volume was brought to 73 μl using the same buffer that was used to resuspend the ANXA1 protein. 27 jd of extrusion generated vesicles were added to each well and vesicle aggregation was measured at wavelength 350 on a SpectraMax M5 96-well plate reader (Molecular Devices, San Jose, Calif.). For calcium chelation, 0.48 mmol/L ethylenediaminetetraacetic acid was incubated with 1 μg human ANXA1 protein for five minutes at room temperature prior to adding the extrusion generated vesicles, after which the reaction was carried out for 20 minutes and then aggregation was determined on a plate reader. For ANXA1 neutralizing antibody assessment, 0.01-1 μg N-terminal ANXA1 antibody (Abcam, #ab33061) designed against a synthetic peptide, MAMVSEFLKQAWFIENEEQEYVQTVKSSKGGPGSAVSPYPTFNPSSDVAA (SEQ ID NO: 25), corresponding to the N-terminal amino acid 1-50 region of human ANXA1, was incubated with 1 μg human ANXA1 protein for five minutes prior to adding the extrusion generated vesicles.

NanoSight Nanoparticle Tracking Analysis. Vesicle diameter and particle abundance was determined by NanoSight™ LM10 (Malvern Instruments, Malvern, United Kingdom) nanoparticle tracking analysis. Five runs were used to calculate the mean and standard deviation for each sample.

Extracellular Vesicle Calcification Assay. 3D-collagen hydrogels were cast as previously described (10). Rat tail collagen type I (Corning, Corning, N.Y.) at a pH 7-8 was used, as in this pH range the collagen forms a hydrogel network. 150 μl collagen solution was added to chambered cover glass wells (Fisher Scientific, LAB-TEK, #1.5 borosilicate) and allowed to solidify in a cell culture incubator for two hours. 150 μl of EVs in day-14 SMC or VIC conditioned media was added to the collagen hydrogels and incubated at 37° C., replacing media with new day-14 EV conditioned media every three days for a total of 21 days. On the day prior to imaging, OsteoSense680 (Perkin Elmer; 1:100) was added to the collagen hydrogels and incubated overnight at 37° C. On the day of imaging, CNA35-0G488 collagen probe (1:400) was added and incubated for 1 hour at 37° C., followed by washing gels in phosphate buffered saline.

Calcification Quantification. EV aggregation-mediated calcification was imaged by confocal microscopy on a confocal microscope A1 (Nikon Instruments Inc.), and all images were processed with Elements 3.20 software (Nikon Instruments Inc.). Structure illumination microscopy (Zeiss ELYRA Super-Resolution) was performed at the Harvard Center for Biological Imaging to image calcification aggregates in the collagen hydrogels. Assessments of calcification and collagen staining were performed using ImageJ (NIH) analysis of confocal-obtained images in both normal and osteogenic conditioned media treatments. First, the image channels were separated by color (red, green) and the intensities of red staining (calcification) or green staining (collagen) were computed by ImageJ as the integral of the red or green channel histogram. Calcification was normalized to the amount of collagen stain in each well for quantification. For SMC and VIC calcification quantification, calcification was imaged and quantified in a similar manner as with EVs, except normalization was made using total cell count quantified by DAPI staining. Investigators were blinded to the treatment group for quantification.

RNA and Protein Analysis. RNA was extracted using TRIzol (Fisher Scientific), and cDNA was made using the qScript cDNA Synthesis Kit (Quanta Biosciences, Gaithersburg, Md.). Quantitative PCR was performed using the following Taqman probes (Fisher Scientific): Hs02758991_g1 (GAPDH); Hs00167549_m1 (ANX4J); Hs00743063_s1 (ANXA2); Hs00996187_m1 (ANXA5); Hs01049082m1 (ANXA6). Fold changes were normalized to GAPDH and determined by the AACt method. Western blot analysis was performed by lysing cells in RIPA buffer (Fisher Scientific) containing EDTA-free protease inhibitor (Sigma), loading 20 μg protein lysate, and using the following antibodies: ANXA1 (Abcam #ab214486; 1:1000), DRP1 (Abcam #ab56788; 1:1000), β-actin (Novus Biologicals LLC, Centennial, Colo., #NB600-501; 1:5000), and a-tubulin (Abcam #ab15246; 1:1000). ANXA1 EVs surface localization was assessed by isolating human SMC and VIC EVs. 10 mL of conditioned media was centrifuged at 100,000 g for 40 minutes, and the resulting EVs pellet was resuspended in PBS or PBS containing 0.48 mmol/L EDTA and incubated for 30 minutes at 37° C. EVs were pelleted a second time at 100,000 g for 40 minutes. The supernatant was collected, and the pellet was resuspended in 100 μl PBS. Samples were incubated with 6×-loading buffer (Boston Bioproducts) and boiled for 10 minutes, prior to analysis by western blot. ANXA1 released into the supernatant was assessed as being on the surface of EVs.

Statistical Analysis. PRISM software (GraphPad, San Diego, CA) was used to analyze data by ANOVA with Tukey's multiple comparisons test or t-test when appropriate. Bar graph data were plotted as mean±standard deviation (s.d.), and individual datapoints were included as dot points. For mass spectrometry analysis P values were adjusted using the Benjamini and Hochberg method (false discovery rate), and the results were visualized using volcano plots.

Illustrations. Illustrations were generated using Microsoft PowerPoint and the Motifolio illustration tool kit (Ellicott City, Md.).

### REFERENCES

1. Caro L G, Palade G E (1964) Protein Synthesis, storage, and discharge in the pancreatic exocrine cell. J Cell Biol 20:473-495.
2. Block M R, Glick B S, Wilcox C A, Wieland F T, Rothman J E (1988) Purification of an N-ethylmaleimide-sensitive protein catalyzing vesicular transport. Proc Natl Acad Sci USA 85:7852-7856.
3. Barlowe C, et al. (1994) COPII: a membrane coat formed by Sec proteins that drive vesicle budding from the endoplasmic reticulum. Cell 77:895-907.
4. Luo F, Liu X, SUdhof TC, Acuna C (2017) Efficient stimulus-secretion coupling at ribbon synapses requires RIM-binding protein tethering of L-type Ca²⁺ channels. Proc Nal Acad Sci USA 114:E8081-E8090.
5. Wickner W T (2013) Profile of Thomas Südhof, James Rothman, and Randy Schekman, 2013 Nobel Laureates in Physiology or Medicine. Proc Natl Acad Sci USA 110:18349-18350.
6. Cao X, Ballew N, Barlowe C (1998) Initial docking of ER-derived vesicles requires Usolp and Yptlp but is independent of SNARE proteins. EMBO J17:2156-2165.
7. Pfeffer S R (1999) Transport-vesicle targeting: tethers before SNARES. Nat Cell Biol 1:E17-22.
8. D'Agostino M, Risselada H J, LUrick A, Ungermann C, Mayer A (2017) A tethering complex drives the terminal stage of SNARE-dependent membrane fusion. Nature 551:634-638.
9. Golub E E (2009) Role of matrix vesicles in biomineralization. Biochim Biophys Acta 1790:1592-1598.
10. Hutcheson J D, et al. (2016) Genesis and growth of extracellular-vesicle-derived microcalcification in atherosclerotic plaques. Nat Mater 15:335-343.
11. Kapustin A N, et al. (2011) Calcium regulates key components of vascular smooth muscle cell-derived matrix vesicles to enhance mineralization. Circ Res 109: e 1-12.
12. Kim K M (1976) Calcification of matrix vesicles in human aortic valve and aortic media. Fed Proc 35:156-162.
13. Kelly-Arnold A, et al. (2013) Revised microcalcification hypothesis for fibrous cap rupture in human coronary arteries. Proc Natl Acad Sci USA 110:10741-10746.
14. Rogers M A, Aikawa E (2019) Cardiovascular calcification: artificial intelligence and big data accelerate mechanistic discovery. Nat Rev Cardiol 16:261-274.
15. Chen N X, O'Neill K D, Chen X, Moe S M (2008) Annexin-mediated matrix vesicle calcification in vascular smooth muscle cells. J Bone Miner Res 23:1798-1805.
16. New S E, et al. (2013) Macrophage-derived matrix vesicles: an alternative novel mechanism for microcalcification in atherosclerotic plaques. Circ Res 113:72-77.
17. Goettsch C, et al. (2016) Sortilin mediates vascular calcification via its recruitment into extracellular vesicles. J Clin Invest 126:1323-1336.
18. Kirsch T, Harrison G, Golub E E, Nah H D (2000) The roles of annexins and types II and X collagen in matrix vesicle-mediated mineralization of growth plate cartilage. J Biol Chem 275:35577.
19. Lee G, Pollard H B (1997) Highly sensitive and stable phosphatidylserine liposome aggregation assay for annexins. Anal Biochem 252:160-164.
20. Smits M (2016) Imaging of oligodendroglioma. Br J Radiol 89:20150857.
21. Lo Cicero A, Maikowska I, Nagase H, Di Liegro I, Troeberg L (2012) Microvesicles shed by oligodendroglioma cells and rheumatoid synovial fibroblasts contain aggrecanase activity. Matrix Biol 31:229-233.
22. Bos D, et al. (2015) Atherosclerotic calcification is related to a higher risk of dementia and cognitive decline. Alzheimers Dement 11:639-647.
23. Dinkins M B, Dasgupta S, Wang G, Zhu G, Bieberich E (2014) Exosome reduction in vivo is associated with lower amyloid plaque load in the SXFAD mouse model of Alzheimer's disease. Neurobiol Aging 35:1792-1800.
24. Steen V D, Ziegler G L, Rodnan G P, Medsger T A Jr (1984) Clinical and laboratory associations of anticentromere antibody in patients with progressive systemic sclerosis. Arthritis Rheum 27:125-131.
25. Maugeri N, et al. (2018) Platelet microparticles sustain autophagy-associated activation of neutrophils in systemic sclerosis. Sci Transl Med 10:eaao3089.
26. Leach R J et al. (1995) Osteosarcoma hybrids can preferentially target alkaline phosphatase activity to matrix vesicles: evidence for independent membrane biogenesis. J Bone Miner Res 10:1614-1624.
27. Kapustin A N, et al. (2015) Vascular smooth muscle cell calcification is mediated by regulated exosome secretion. Circ Res 116:1312-1323.
28. Schlotter F, et al. (2018) Spatiotemporal multi-omics mapping generates a molecular atlas of the aortic valve and reveals networks driving disease. Circulation 138:377-393.
29. Eden E R, et al. (2016) Annexin A1 tethers membrane contact sites that mediate ER to endosome cholesterol transport. Dev Cell 37:473-483.
30. Rogers M A, et al. (2017) Dynamin-related protein 1 inhibition attenuates cardiovascular calcification in the presence of oxidative stress. Circ Res 121:220-233.
31. Draeger A, Wray S, Babiychuk E B (2005) Domain architecture of the smooth-muscle plasma membrane: regulation by annexins. Biochem J387:309-314.
32. Bode G, et al. (2008) Interaction between S100A8/A9 and annexin A6 is involved in the calcium-induced cell surface exposition of S100A8/A9. J Biol Chem 283:31776-31784.
33. Cui L, et al. (2017) End stage renal disease-induced hypercalcemia may promote aortic valve calcification via Annexin VI enrichment of valve interstitial cell derived-matrix vesicles. J Cell Physiol 232:2985-2995.
34. Aikawa E, et al. (2007) Osteogenesis associates with inflammation in early-stage atherosclerosis evaluated by molecular imaging in vivo. Circulation 116:2841-2850.
35. Aper S J, et al. (2014) Colorful protein-based fluorescent probes for collagen imaging. PLoS One 9:e114983.
36. Eqi M, et al. (2011) Ionized calcium concentration and outcome in critical illness. Crit Care Med 39:314-321.
37. Soares Martins T, et al. (2018) Exosome isolation from distinct biofluids using precipitation and column-based approaches. PLoS One 13:e0198820.
38. Mirsaeidi M, Gidfar S, Vu A, Schraufnagel D J (2016) Annexins family: insights into their functions and potential role in pathogenesis of sarcoidosis. J Transl Med 14:89.
39. Pandey S, et al. (2012) Effect of ANXA2 gene single nucleotide polymorphism (SNP) on the development of osteonecrosis in Indian sickle cell patient: a PCR-RFLP approach. Indian J Exp Biol 50:455-458.
40. Miyamura H, et al. (2011) Polymorphisms in the annexin A5 gene promoter in Japanese women with recurrent pregnancy loss. Mol Hum Reprod 17:447-452.
41. Rodriguez-Garcia M I, et al. (1996) Annexin V autoantibodies in rheumatoid arthritis. Ann Rheum Dis 55:895-900.
42. Rong B, et al. (2014) Elevated serum annexin A1 as potential diagnostic marker for lung cancer: a retrospective case-control study. Am J Trans! Res 6:558-569.
43. Boisserie-Lacroix et al. (2012) Radiological features of triple-negative breast cancers (73 cases). Diagn and Intery Imaging 93:183-190.
44. Okano M, et al. (2015) Upregulated annexin A1 promotes cellular invasion in triple-negative breast cancer. Oncol Rep 33:1064-1070.
45. Peters M E M, et al. (2018) Histological validation of calcifications in the human hippocampus as seen on computed tomography. PLoS One 13:e0197073.
46. Ries M, et al. (2015) The anti-inflammatory annexin A1 induces the clearance and degradation of the amyloid-β peptide. J Neuroinflammation 13:234.
47. Edgar J R, Manna PT, Nishimura S, Banting G, Robinson M S (2016) Tetherin is an exosomal tether. Elife 5:e17180.
48. Dai X Y, et al. (2013) Phosphate-induced autophagy counteracts vascular calcification by reducing matrix vesicle release. Kidney Int 83:1042-1051.
49. Fredman G, et al. (2015) Targeted nanoparticles containing the proresolving peptide Ac2-26 protect against advance atherosclerosis in hypercholesterolemic mice. Sci Transl Med 7:275ra20.
50. Weinberger A, et al. (2013) Gel-assisted formation of giant unilameller vesicles. Biophys J 105:154-164.

### Example 3

The present disclosure features methods for the reduction of vascular and valvular calcification through administration of Annexin A1 inhibitors. Annexin A1 inhibitors are used to reduce cardiovascular calcification by modulation of extravellular vesicle tethering and tissue non-specific alkaline phosphatase activity.

Extracellular vesicles (EVs) contribute to extracellular matrix calcification /microcalcification that can lead to plaque rupture (Kelley-Arnold PNAS 2013). EVs aggregate, nucleate hydroxyapatite and form microcalcifications (Hutcheson et al. Nature Materials 2016). However, how they aggregate is unknown. It was hypothesized that the membranes of EVs contain proteins with tethering functions that induce vesicle aggregation and promote microcalcification in collagen rich matrices.

Calcified tissue was collected from patients undergoing heart valve replacement (N=3) and carotid endarterectomy procedures (N=3), and electron microscopy was used to observe vesicles within diseased tissue. Calcified tissue was also taken from patients undergoing heart valve replacement (N=5) and carotid endarterectomy (N=5) proceedurers and stained for Annexin A1 by immunohistochemistry. Primary human cardiovascular cells were cultured in osteogenic differentiation media and cell protein lysate was collected for alkaline phosphotase activity assaying. EVs were isolated from primary human smooth muscle cells (N=9) and valvular interstitial cells (N=3) cultured in osteogenic differentiation media, and mass spectrometry was used along with network analysis to identify proteins associated with membrane tethering. GFP-tagged candidate proteins where purified and incubated with protein-free vesicles and assessed for membrane tethering activity.

siRNA was used to knockdown candidate proteins in primary human cardiovascular cells, and EVs were incubated in cell-free three-dimensional collagen hydrogels that mimic structural features of the atherosclerotic fibrous cap to assess vesicular calcification potential using Osteosense imaging. Calcified human cardiovascular tissues contained aggregated vesicles, and both the tissues (assesed by immunohistochemistry) and the tissue vesicles (assessed by immunogold electron microscopy) stained positive for Annexin A1. Mass spectrometry and network analysis identified Annexin A1 as a membrane tethering protein enriched in EVs from osteogenic media treated primary human cardiovascular cells. Annexin A1 siRNA ameliorated EV-mediated calcification in three-dimensional collagen hydrogels and attenuated the induction of alkaline phosphotase activity in primary human cardiovascular cells (N=3).

It was found that vesicle membrane proteins, specifically Annexin A1, function to aggregate EVs, providing an explanation for the origin of cardiovascular calcification formation from sub-micromolar-sized vesicles. Collagen rich matrices can trap EVs released from cardiovascular cells that then aggregate via membrane tethering proteins on the vesicle surface. The role of Annexins in calcification may be more complex than previously thought, with Annexin A1 having a novel function in vesicle tethering and initiating the calcification process.

Described herein is a novel method for reducing vascular and valvular calcification. This is the first report to demonstrate a role of Annexin A1 in the regulation of cardiovascular calcification, extracellular vesicle tethering, and cardiovascular cell alkaline phosphotase activity. While other Annexins including A2, A5, and A6 have been previously reported to play a role in extracellular vesicle calcification, perhaps through calcium binding, no studies have reported a role for Annexin A1 in cardiovascular calcification. Additionally, no reports have been made of a method to prevent extracellular vesicle aggregation by inhibiting vesicle tethering or any other means. Herein is provided a novel means to inhibit vascular and valvular calcification through Annexin A1 inhibition, which leads to reduced vesicle tethering and aggregation, reduced alkaline phosphotase activity, and attentuation of vesicle-mediated calcification

Inhibitors can be produced to suppress/modulate the function of Annexin A1 and be used to reduce vascular and valvular calcification. Non-limiting examples of Annexin A1 inhibitors include small molecules that modulate Annexin A1 activity, binding, or localization, along with molecules that can inhibit Annexin A1 expression, e.g., siRNA, antisense molecules, or gene editing technologies. The methods described herein include administering to a patient an effective amount of Annexin A1 inhibitor in a pharmaceutically acceptable vehicle. The Annexin A1 inhibitor is admistered alone or in combination with one or more other therapies.

### Example 4

Extracellular vescicles (EVs) contribution to extracellular matrix calcification/microcalcification that can lead to plaque rupture. It is demonstrated herei that EV membranes contain proteins with tethering functions that induce vesicle aggregation and promote calcification in collagen rich matrices.

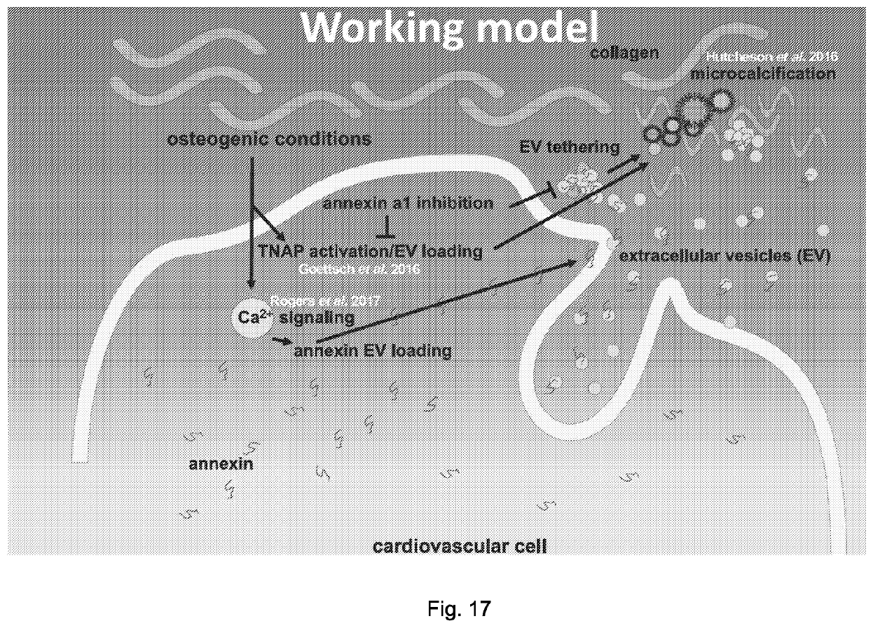

Collagen rich matrices may trap EVs releated from cardiovascular cells that then aggregate via membrane tethering proteins on the vesicle surface. The role of Annexins in calcification is more complex than previously thought, as Annexin A1 (ANXA1) functions as a vesicle tether and TNAP regulator initiating the calcification process. FIG. 17 depicts a working model of this role of ANXA1.

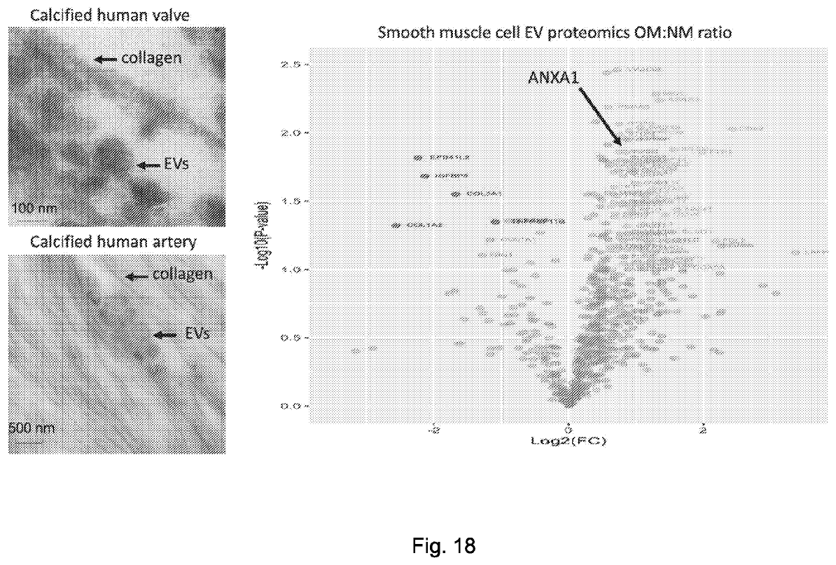

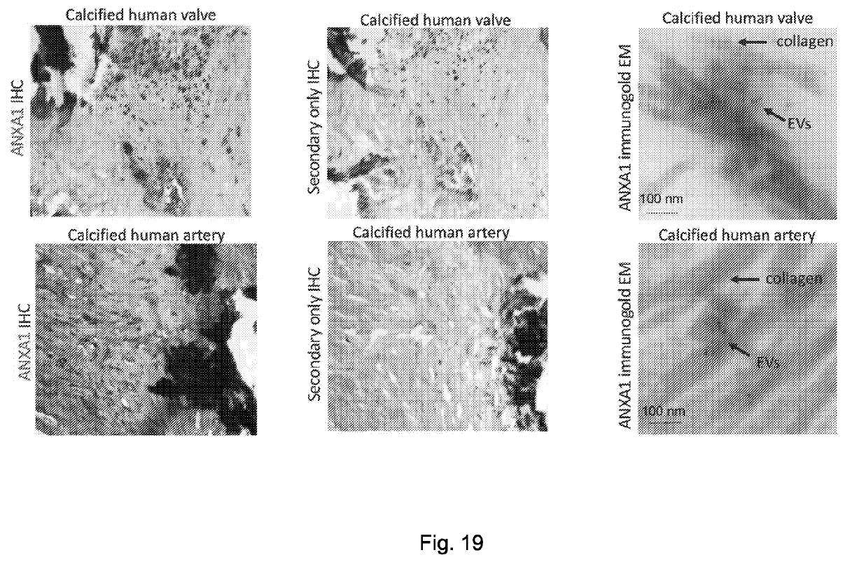

EVs aggregate in calcified human tissues and EV ANXA1 is increased by osteogenic conditions (FIG. 18). Calcified human tissues and vesicles contain ANXA1 (FIG. 19).

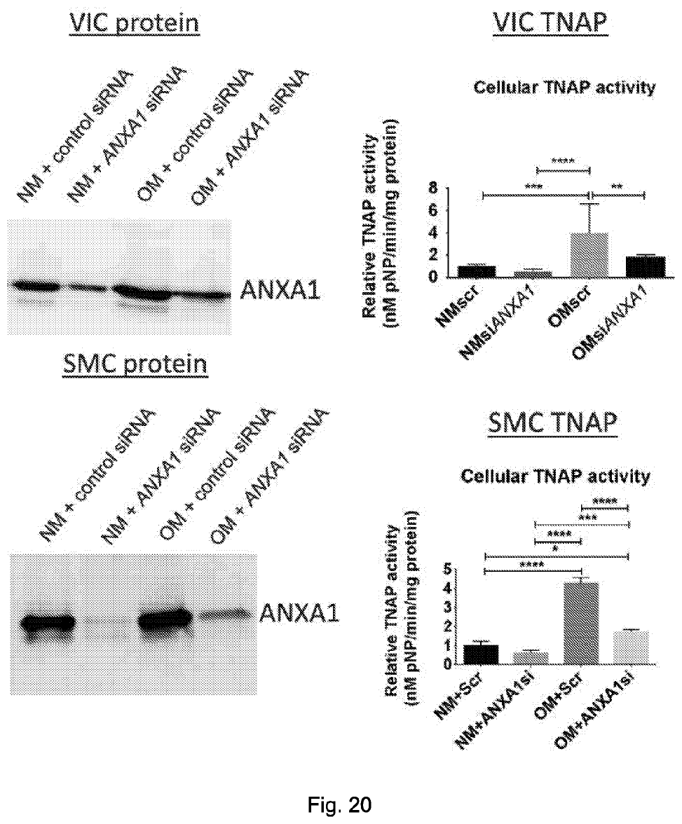

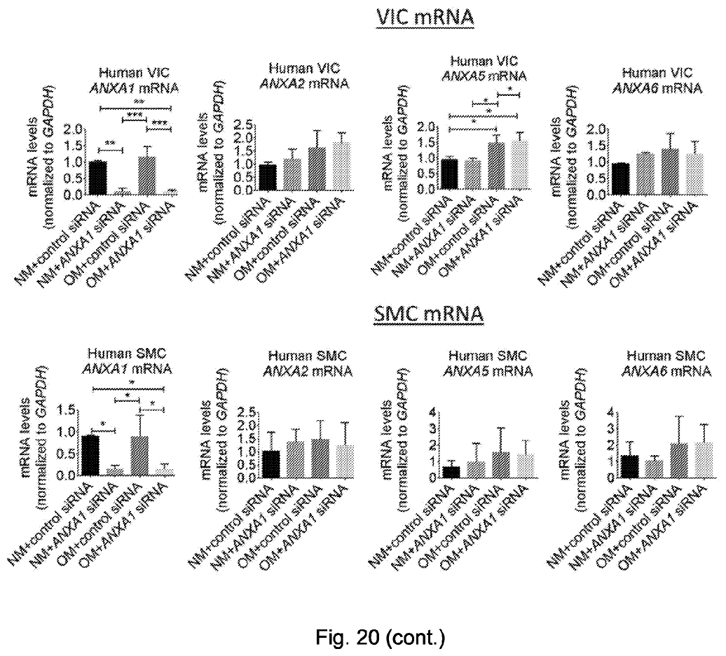

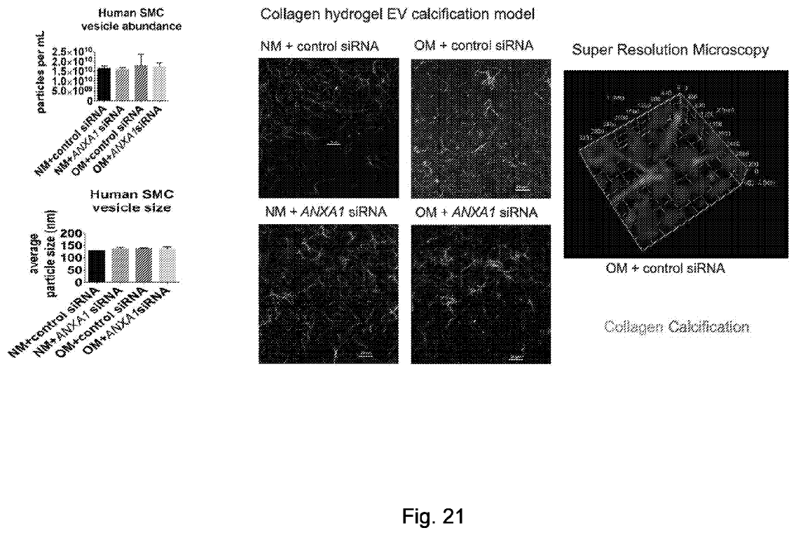

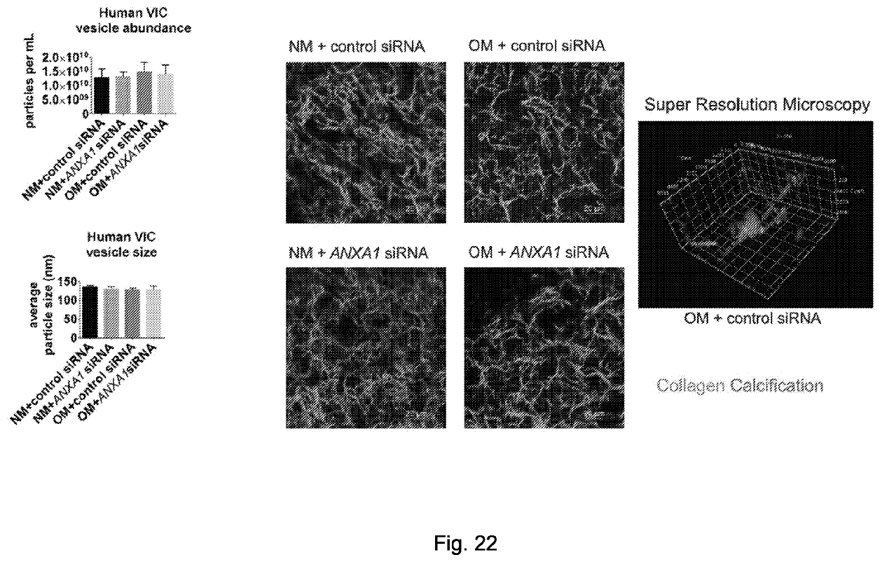

ANXA1 siRNA reduced osteogenic media-induced TNAP activity in human VICs and SMCs (FIG. 20) and suppressed human SMC vesicle-mediated calcification (FIG. 21) as well as human VIC vesicle-mediated calcification (FIG. 22).

1. A method of treating an extracellular vesicle (EV)-associated disease in subject, the method comprising: administering an agent that reduces the levels or activity of Annexin A1 (ANXA1) in a subject in need thereof.

2. The method of claim 1, wherein the EV-associated disease is selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.

3. A method of reducing vascular calcification in subject, the method comprising: administering an agent that reduces the levels or activity of Annexin A1 (ANXA1) in a subject in need thereof.

4. The method of claim 3, wherein the subject has a disease selected from the group consisting of: valvular heart disease, vascular disease, rheumatoid arthritis, neurodegenerative disease, autoimmune disease, calcific aortic valve disease, diabetes, systemic lupus erythematosus, ulcerative colitis, pulmonary fibrosis, non-alcoholic fatty liver disease, osteoporosis, Alzheimer's disease, scleroderma, atherosclerosis, myocardial infarction, hypercholesterolemia, cancer, and obesity.

5. A method for inhibiting or reducing trafficking of an extracellular vesicle (EV) from a cell, the method comprising: contacting the cell with an agent that reduces the levels or activity of ANXA1.

6. The method of claim 5, wherein the cell is a smooth muscle cell (SMC), a valvular interstitial cell (VIC), oligodendroglioma cell, endothelial cell, macrophage, monocyte, or cancer cell.

7. The method of claim 1, wherein the agent is an inhibitor of ANXA1.

8. The method of claim 1, wherein the agent is a small molecule, nucleic acid, polypeptide, antibody reagent, or genome editing system.

9. The method of claim 8, wherein the nucleic acid is an inhibitory nucleic acid, silencing RNA (siRNA), microRNA (miRNA), or short hairpin RNA (shRNA).

10. The method of claim 9, wherein the siRNA sequence comprises SEQ ID NO: 1 or 2.

11. The method of claim 8, wherein the antibody reagent is an anti-ANXA1 antibody reagent.

12. The method of claim 11, wherein the antibody reagent is an ANXA1 neutralizing antibody reagent.

13. The method of claim 12, wherein the ANXA1 neutralizing antibody reagent is an N-terminal ANXA1 neutralizing antibody reagent.

14. The method of claim 8, wherein the small molecule is a calcium chelator.

15. The method of claim 14, wherein the calcium chelator is ethylenediaminetetraacetic acid (EDTA).

16. The method of claim 1, wherein the agent reduces ANXA1 loading into the extracellular vesicles.

17. The method of claim 16, wherein the agent is an inhibitor of dynamin-related protein 1 (DRP1).

18. The method of claim 17, wherein the agent is mdivi-1 or is an inhibitory nucleic acid.

19. The method of claim 1, wherein the agent induces cleavage of the N-terminal domain of ANXA1.

20. The method of claim 19, wherein the agent is an agonist of proteinase 3 or HLE.

21.-51. (canceled)

52. The method of claim 1, wherein the subject is a subject determined to have an increased level or activity of ANXA1.


SEQUENCE LISTING (25 sequences)

SEQ ID NO: 1 - DNA, 6656 bp, Homo sapiens
gcaggtgacccggaggccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 2 - DNA, 1401 bp, Homo sapiens
agtgtgaaatcttcagagaagaatttctctttagttctttgcaagaaggtagagataaagacactttttcaaaaatggcaatggtatcagaattcctcaagcaggcctggtttattgaaaatgaagagcaggaatatgttcaaactgtgaagtcatccaaaggtggtcccggatcagcggtgagcccctatcctaccttcaatccatcctcggatgtcgctgccttgcataaggccataatggttaaaggtgtggatgaagcaaccatcattgacattctaactaagcgaaacaatgcacagcgtcaacagatcaaagcagcatatctccaggaaacaggaaagcccctggatgaaacactgaagaaagcccttacaggtcaccttgaggaggttgttttagctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggtgtgaatgaagacttggctgattcagatgccagggccttgtatgaagcaggagaaaggagaaaggggacagacgtaaacgtgttcaataccatccttaccaccagaagctatccacaacttcgcagagtgtttcagaaatacaccaagtacagtaagcatgacatgaacaaagttctggacctggagttgaaaggtgacattgagaaatgcctcacagctatcgtgaagtgcgccacaagcaaaccagctttctttgcagagaagcttcatcaagccatgaaaggtgttggaactcgccataaggcattgatcaggattatggtttcccgttctgaaattgacatgaatgatatcaaagcattctatcagaagatgtatggtatctccctttgccaagccatcctggatgaaaccaaaggagattatgagaaaatcctggtggctctttgtggaggaaactaaacattcccttgatggtctcaagctatgatcagaagactttaattatatattttcatcctataagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagacaa

SEQ ID NO: 3 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 4 - DNA, 1401 bp, Homo sapiens; identical to SEQ ID NO: 2

SEQ ID NO: 5 - DNA, 909 bp, Homo sapiens
agagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgta

SEQ ID NO: 6 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 7 - DNA, 987 bp, Homo sapiens
ggaccccaccatggctcaccggccccccagccctgccctggcgtccgtgctgctggccttgctgctgagcggtgctgcccgagctgcggagatcgtgggcgggcacgaggcgcagccacactcccggccctacatggcctccctgcagatgcgggggaacccgggcagccacttctgcggaggcaccttgatccaccccagcttcgtgctgacggccgcgcactgcctgcgggacataccccagcgcctggtgaacgtggtgctcggagcccacaacgtgcggacgcaggagcccacccagcagcacttctcggtggctcaggtgtttctgaacaactacgacgcggagaacaaactgaacgacgttctcctcatccagctgagcagcccagccaacctcagtgcctccgtcgccacagtccagctgccacagcaggaccagccagtgccccacggcacccagtgcctggccatgggctggggccgcgtgggtgcccacgaccccccagcccaggtcctgcaggagctcaatgtcaccgtggtcaccttcttctgccggccacataacatttgcactttcgtccctcgccgcaaggccggcatctgcttcggagactcaggtggccccctgatctgtgatggcatcatccaaggaatagactccttcgtgatctggggatgtgccacccgccttttccctgacttcttcacgcgggtagccctctacgtggactggatccgttccacgctgcgccgtgtggaggccaagggccgcccctgaaccgcccctcccacagcgctggccgggaccccgagcctggctccaaaccctcgaggcggatctttggacagaagcagctcttccccgaacactgtggcgtccgggacggccccacccgtccccccacactccctcccacggggctccgggagacaggccggccctgcacctcaccccaccgtgacctcaataaacgttgaaactcc

SEQ ID NO: 8 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 9 - protein, 725 aa, Homo sapiens
MEALIPVINKLQDVFNTVGADIIQLPQIVVVGTQSSGKSSVLESLVGRDLLPRGTGIVTRRPLILQLVHVSQEDKRKTTGEENGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 10 - protein, 749 aa, Homo sapiens
MEALIPVINKLQDVFNTVGADIIQLPQIVVVGTQSSGKSSVLESLVGRDLLPRGTGIVTRRPLILQLVHVSQEDKRKTTGEENDPATWKNSRHLSKGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKLIQDSRRETKNVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 11 - protein, 738 aa, Homo sapiens
MEALIPVINKLQDVFNTVGADIIQLPQIVVVGTQSSGKSSVLESLVGRDLLPRGTGIVTRRPLILQLVHVSQEDKRKTTGEENDPATWKNSRHLSKGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 12 - protein, 533 aa, Homo sapiens
MFHKKINGKQQEKKMTLLHGKTQDTFLKGWKQKNGVNFFTPKIRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKLIQDSRRETKNVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 13 - protein, 712 aa, Homo sapiens
MEALIPVINKLQDVFNTVGADIIQLPQIVVVGTQSSGKSSVLESLVGRDLLPRGTGIVTRRPLILQLVHVSQEDKRKTTGEENDPATWKNSRHLSKGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 14 - protein, 699 aa, Homo sapiens
MEALIPVINKLQDVFNTVGADIIQLPQIVVVGTQSSGKSSVLESLVGRDLLPRGTGIVTRRPLILQLVHVSQEDKRKTTGEENGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 15 - protein, 736 aa, Homo sapiens
MEALIPVINKLQDVFNTVGADIIQLPQIVVVGTQSSGKSSVLESLVGRDLLPRGTGIVTRRPLILQLVHVSQEDKRKTTGEENGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKLIQDSRRETKNVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 16 - protein, 710 aa, Homo sapiens
MEALIPVINKLQDVFNTVGADIIQLPQIVVVGTQSSGKSSVLESLVGRDLLPRGTGIVTRRPLILQLVHVSQEDKRKTTGEENGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKLIQDSRRETKNVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 17 - DNA, 4582 bp, Homo sapiens
agcgcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaagtcttctaaagttccaagtgctttggcacctgcctcccaggagccctcccccgctgcttctgctgaggctgatggcaaggttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 18 - DNA, 4654 bp, Homo sapiens
agcgcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatgaccctgctacatggaaaaactcaagacacctttctaaaggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaagtcttctaaagttccaagtgctttggcacctgcctcccaggagccctcccccgctgcttctgctgaggctgatggcaagttaattcaggacagcagaagagaaactaaaaatgttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 19 - DNA, 4621 bp, Homo sapiens
agcgcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatgaccctgctacatggaaaaactcaagacacctttctaaaggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaagtcttctaaagttccaagtgctttggcacctgcctcccaggagccctcccccgctgcttctgctgaggctgatggcaaggttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 20 - DNA, 4211 bp, Homo sapiens
agcgcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatgaccctgctacatggaaaaactcaagacacctttctaaaggggtggaagcagaagaatggggtaaatttcttcacaccaaaaataaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaagtcttctaaagttccaagtgctttggcacctgcctcccaggagccctcccccgctgcttctgctgaggctgatggcaagttaattcaggacagcagaagagaaactaaaaatgttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 21 - DNA, 4543 bp, Homo sapiens
agcgcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatgaccctgctacatggaaaaactcaagacacctttctaaaggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaaggttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 22 - DNA, 4504 bp, Homo sapiens
agcgcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaaggttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 23 - DNA, 4514 bp, Homo sapiens
gtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaagtcttctaaagttccaagtgctttggcacctgcctcccaggagccctcccccgctgcttctgctgaggctgatggcaagttaattcaggacagcagaagagaaactaaaaatgttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 24 - DNA, 4537 bp, Homo sapiens
agcgcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaagttaattcaggacagcagaagagaaactaaaaatgttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 25 - protein, 50 aa, Artificial Sequence
  Description of Artificial Sequence Synthetic polypeptide
MAMVSEFLKQAWFIENEEQEYVQTVKSSKGGPGSAVSPYPTFNPSSDVAA